(19) United States
(12) Patent Application Publication
(10) Pub. No.: US 2022/0041894 A1
(43) Pub. Date: Feb. 10, 2022

(54) ROLL PRODUCT WITH CUT LINES

(71) Applicant: 3M INNOVATIVE PROPERTIES COMPANY, SAINT PAUL, MN (US)

(72) Inventors: YASUHIRO KINOSHITA, TOKYO (JP); FUMISHI SATO, HIGASHINE-CITY (JP); KOSUKE MATSUKI, KAHOKU-TOWN (JP)

(73) Assignee: 3M INNOVATIVE PROPERTIES COMPANY, SAINT PAUL, MN (US)

(21) Appl. No.: 17/276,179

(22) PCT Filed: Sep. 20, 2019
(86) PCT No.: PCT/IB2019/057979

§ 371 Date: Mar. 15, 2021

(30) Foreign Application Priority Data
Sep. 25, 2018 (JP) 2018-179052

(51) Int. Cl. C09J 7/20; A61L 24/06; B65H 18/28

(52) U.S. Cl. CPC C09J 7/20; A61F 15/002; B65H 18/28; A61L 24/06

(57) ABSTRACT

The roll product includes an arrangement of actual cut lines to improve hand-tearability and strength. Between the actual cut lines are virtual cut lines. A set of actual cut lines and virtual cut lines forms a cutting induction portion. A plurality of rows of virtual cut lines are arranged in a width direction in each of the cutting induction portions and are formed in a longitudinal direction of the roll product.

### TECHNICAL FIELD

The present disclosure relates to a roll product with an arrangement of cut lines

### BACKGROUND ART

Adhesive coated substrates are used in many applications. One common way of packing adhesive coated substrates is to form a roll product. Patent Document JP 9-502111 T describes a roll product in which a band-shaped member including a strip-shaped base member and a bonding layer formed on one surface of the base material is wound in a roll shape where the roll product has a cut line of perforations formed in the band-shaped member.

### SUMMARY OF THE INVENTION

In a roll product of a band-shaped member, it is desirable for a user to easily tear the band-shaped member by hand. On the other hand, it is desired that the band-shaped member be difficult to tear even when the band-shaped member is pulled in a longitudinal direction. Thus, both improvement in hand-tearability and strength of the band-shaped member have been desired. Disclosed is roll product of a band-shaped member with an arrangement of cut lines to provide hand-tearability and strength of the band-shaped member

In one embodiment, the roll product comprises a band-shaped member including a strip-shaped base member and a bonding layer formed on one surface of the base material wound in a roll shape. A plurality of cutting induction portions extending in a width direction of the band-shaped member are formed in the band-shaped member in a longitudinal direction of the band-shaped member; each of the plurality of cutting induction portions includes a combination of a plurality of actual cut lines arranged to be spaced apart from each other in the width direction, and formed by partially cutting the base member, and a plurality of virtual cut lines set by connecting ends of the plurality of actual cut lines; the plurality of virtual cut lines include a virtual cut line extending from a first end of a first actual cut line of the plurality of actual cut lines and connected to one of ends of a second actual cut line different from the first actual cut line, and the one of the ends of the second actual cut line is closest to the first end of the ends of the second actual cut line; the plurality of virtual cut lines include a virtual cut line extending from a second end of the first actual cut line and connected to one of ends of a third actual cut line different from the first actual cut line and the second actual cut line, and the one of the ends of the third actual cut line is closest to the second end of the ends of the third actual cut line; a plurality of rows of the virtual cut lines arranged in the width direction are formed in the longitudinal direction in each of the cutting induction portions; when a first projection reference line extending in the longitudinal direction is set, and a plurality of first projected portions are set by projecting the plurality of virtual cut lines in each of the cutting induction portions onto the first projection reference line, the first projected portions of the plurality of virtual cut lines belonging to the same row overlap each other, and the first projected portions of the plurality of the virtual cut lines belonging to one row and the first projected portions of the plurality of the virtual cut lines belonging to another row are spaced apart in the longitudinal direction without overlapping each other; and when a second projection reference line extending in the width direction is set, and a plurality of second projected portions are set by projecting the plurality of virtual cut lines in each of the cutting induction portions onto the second projection reference line, the plurality of second projected portions are spaced apart in the width direction without overlapping each other.

### BRIEF DESCRIPTION OF THE DRAWINGS

FIG. 1 is a side view of a roll product according to an embodiment.

FIGS. 2A and 2B are plan views of a tape.

FIG. 3 is an enlarged perspective view of a tape.

FIGS. 4A and 4B are enlarged views of a cutting induction portion of a tape of a roll product according to the embodiment.

FIG. 5 is an enlarged view of a cutting induction portion of a tape of a roll product according to a comparative example.

FIGS. 6A to 6C are enlarged views of a cutting induction portion of a tape of a roll product according to a comparative example.

FIGS. 7A and 7B are enlarged views of a cutting induction portion of a tape of a roll product according to a modified example.

FIG. 8 is an enlarged view of a cutting induction portion of a tape of a roll product according to a modified example.

FIGS. 9A and 9B are enlarged views of a cutting induction portion of a tape of a roll product according to a modified example.

FIGS. 10A and 10B are enlarged views of a cutting induction portion of a tape of a roll product according to a modified example.

FIG. 11 is a table showing conditions and experimental results of examples and comparative examples.

FIG. 12 is a table showing experimental results of examples and comparative examples.

FIGS. 13A and 13B are views illustrating contents of a tearing test.

FIGS. 14A and 14B are views corresponding to the tape including the cutting induction portion illustrated in FIGS. 4A and 4B.

FIGS. 15A to 15D are views corresponding to the tape including the cutting induction portion illustrated in FIGS. 4A and 4B.

FIGS. 16A and 16B are views corresponding to the tape including the cutting induction portion illustrated in FIGS. 4A and 4B.

FIG. 17 is a view corresponding to the tape including the cutting induction portion illustrated in FIGS. 4A and 4B.

FIG. 18 is a view corresponding to the tape including the cutting induction portion illustrated in FIGS. 4A and 4B.

FIGS. 19A and 19B are views corresponding to the tape including the cutting induction portion illustrated in FIG. 8.

FIGS. 20A to 20D are views corresponding to the tape including the cutting induction portion illustrated in FIG. 8.

FIGS. 21A and 21B are views corresponding to the tape including the cutting induction portion illustrated in FIG. 8.

FIGS. 22A and 22B are views corresponding to the tape including the cutting induction portion illustrated in FIG. 8.

FIGS. 23A and 23B are views corresponding to the tape including the cutting induction portion illustrated in FIG. 8.

FIG. 24 is a view corresponding to the tape including the cutting induction portion illustrated in FIG. 8.

FIG. 25 is a view corresponding to the tape including the cutting induction portion illustrated in FIG. 8.

FIGS. 26A and 26B are views corresponding to the tape including the cutting induction portion illustrated in FIG. 7B.

FIGS. 27A and 27B are views corresponding to the tape including the cutting induction portion illustrated in FIG. 7B.

FIGS. 28A and 28B are views corresponding to the tape including the cutting induction portion illustrated in FIG. 7B.

FIGS. 29A and 29B are views corresponding to the tape including the cutting induction portion illustrated in FIG. 7B.

FIG. 30 is a view corresponding to the tape including the cutting induction portion illustrated in FIG. 7B.

FIG. 31 is a view corresponding to the tape including the cutting induction portion illustrated in FIG. 7B.

FIGS. 32A and 32B are views corresponding to the tape including the cutting induction portion illustrated in FIG. 7A.

FIGS. 33A and 33B are views corresponding to the tape including the cutting induction portion illustrated in FIG. 7A.

FIGS. 34A and 34B are views corresponding to the tape including the cutting induction portion illustrated in FIG. 7A.

FIGS. 35A and 35B are views corresponding to the tape including the cutting induction portion illustrated in FIG. 7A.

FIG. 36 is a view corresponding to the tape including the cutting induction portion illustrated in FIG. 7A.

FIG. 37 is a view corresponding to the tape including the cutting induction portion illustrated in FIG. 7A.

FIGS. 38A and 38B are views corresponding to the tape including the cutting induction portion illustrated in FIG. 10A.

FIGS. 39A and 39B are views corresponding to the tape including the cutting induction portion illustrated in FIG. 10A.

FIGS. 40A and 40B are views corresponding to the tape including the cutting induction portion illustrated in FIG. 10A.

FIGS. 41A and 41B are views corresponding to the tape including the cutting induction portion illustrated in FIG. 10A.

FIG. 42 is a view corresponding to the tape including the cutting induction portion illustrated in FIG. 10A.

FIG. 43 is a view corresponding to the tape including the cutting induction portion illustrated in FIG. 10A.

FIGS. 44A and 44B are views corresponding to the tape including the cutting induction portion illustrated in FIG. 10B.

FIGS. 45A and 45B are views corresponding to the tape including the cutting induction portion illustrated in FIG. 10B.

FIGS. 46A and 46B are a view corresponding to the tape including the cutting induction portion illustrated in FIG. 10B.

FIGS. 47A and 47B are views corresponding to the tape including the cutting induction portion illustrated in FIG. 10B.

FIG. 48 is a view corresponding to the tape including the cutting induction portion illustrated in FIG. 10B.

FIG. 49 is a view corresponding to the tape including the cutting induction portion illustrated in FIG. 10B.

While the above-identified drawings forth embodiments of the invention, other embodiments are also contemplated, as noted in the discussion. In all cases, this disclosure presents the invention by way of representation and not limitation. Numerous other modifications and embodiments can be devised by those skilled in the art, which fall within the scope and spirit of this invention. The figures may not be drawn to scale.

### DESCRIPTION OF EMBODIMENTS

Embodiments of the present invention will be described in detail below with reference to the accompanying drawings. In the description with reference to the drawings, identical elements will be denoted by identical signs, and redundant descriptions will be omitted.

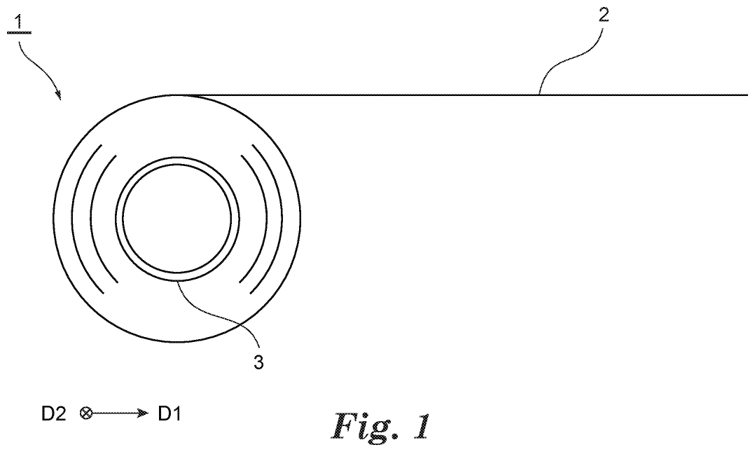

First, a configuration of a roll product 1 will be described with reference to FIG. 1. The roll product 1 is a product including a tape 2 (band-shaped member) wound in a roll shape. The roll product 1 includes the tape 2 continuously wound around a shaft member 3 multiple times. In use, the tape 2 of a desired length is used by unwinding the tape 2 from the roll product 1 and cutting the tape 2. The tape 2 of the roll product 1 is used as a tape for medical use, athletic use, or the like.

Note that in the following description, a direction in which the tape 2 extends is referred to as a “longitudinal direction D1,” and a direction that is a plane direction of the tape 2 and is orthogonal to the longitudinal direction D1 is referred to as a “width direction D2.” In the drawings, the positive side of the longitudinal direction D1 indicates the “unwinding side” to which the tape 2 is unwound, and the negative side of the longitudinal direction D1 indicates the “rewinding side” to which the tape 2 is rewound. The positive side of the width direction D2 indicates the side from one end 2c toward the other end 2d of the tape 2, and the negative side of the width direction D2 indicates the side from the other end 2d toward the one end 2c of the tape 2. Although a dimension in the width direction D2 of the tape 2 may be changed appropriately in accordance with the use and the like, the dimension may be set within the range from, for example, 10 to 100 mm and the like.

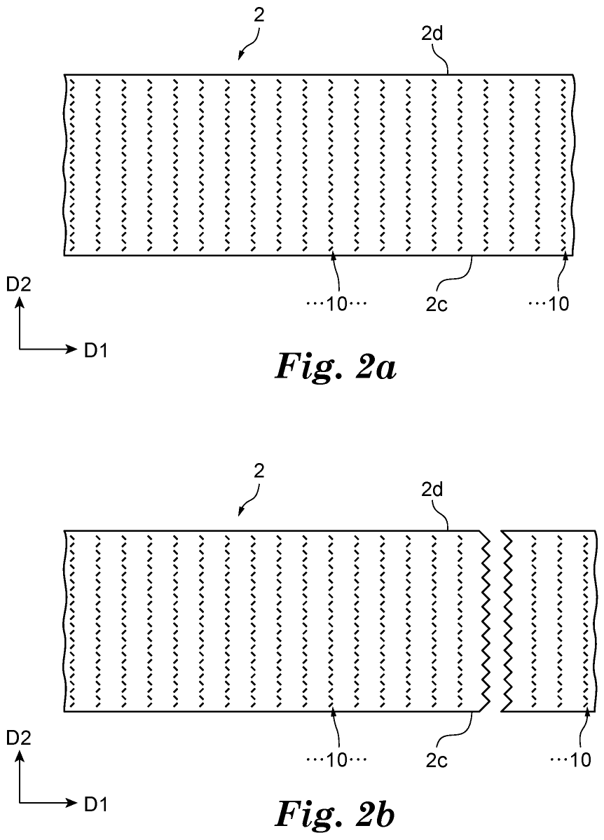

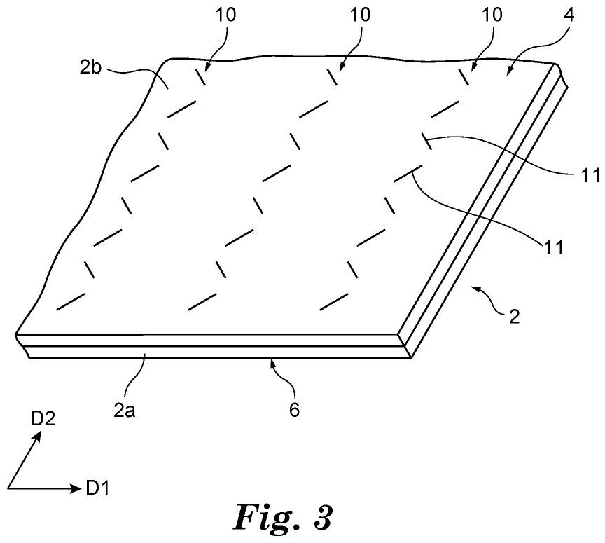

As illustrated in FIGS. 2A to 3, a plurality of cutting induction portions 10 extending in the width direction D2 are formed in the longitudinal direction D1 in the tape 2. The cutting induction portions 10 extend across an entire region between the end 2c and the end 2d in the width direction D2 of the tape 2. A pitch in the longitudinal direction D1 of the cutting induction portions 10 may be set to, but not particularly limited to, the range from 2 to 100 mm and the like. The cutting induction portions 10 are formed at an even pitch in the longitudinal direction D1 but may not be formed at an even pitch. In a region between one cutting induction portion 10 and another cutting induction portion 10 adjacent in the longitudinal direction D1 to the one cutting induction portion 10, no cut line or the like is provided and nonwoven fabric continuously extends in the longitudinal direction D1 and the width direction D2. When a user tears the tape 2 by hand, the cutting induction portion 10 induces a cut at a position of the cutting induction portion 10. That is, the cutting induction portion 10 is a location where strength against the tearing force is reduced and the location easily becomes a cut line after cutting in comparison with locations where the nonwoven fabric continuously extends in the longitudinal direction D1 and the width direction D2. Details of the configuration of each cutting induction portion 10 will be described below.

As illustrated in FIG. 3, the tape 2 includes a strip-shaped base member 4 and a bonding layer 6 formed on one surface of the base member 4. The base member 4 can be a formed of various materials, such as, films, paper, nonwoven fabrics, an can be a single layer or multilayer. For example in one embodiment, the base member is a nonwoven fabric and may further be covered with a resin layer of polyester, urethane, acrylic or the like. A material of the nonwoven fabric constituting the base member 4 may be, but is not limited to, rayon, cellulose, polyester, polyurethane, polyolefin or the like. The bonding layer 6 is provided by forming a layer of an adhesive or a glue on one surface of the base member 4. A material of the bonding layer 6 may be, but not limited to, an acrylic material, a silicone material, a rubber material, or a urethane material in consideration of ease of application to the skin of the human body, for example. Accordingly, the tape 2 is configured as a tape having bonding force to a target object on one surface 2a and having no bonding force to the target object on the other surface 2b.

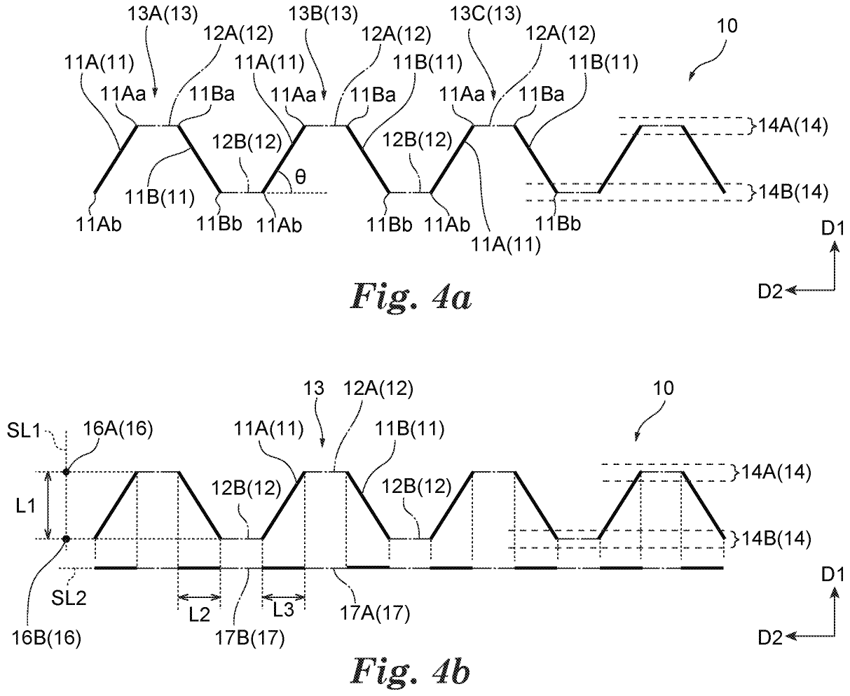

Next, a configuration of each cutting induction portion 10 will be described with reference to FIGS. 4A and 4B. As illustrated in FIG. 4A, the cutting induction portion 10 includes a combination of a plurality of actual cut lines 11 and a plurality of virtual cut lines 12. Note that a “virtual cut line,” a “projection reference line” and a “projected portion” mentioned in the description are not set in a visually recognizable manner to the tape 2 but are virtually set for the purpose of identifying a configuration of each component.

The actual cut lines 11 are spaced apart from each other in the width direction D2 and are formed by partially cutting the base member 4. The actual cut lines 11 are locations having been partially cut in advance at the time before a user tears the tape 2. In the actual cut lines 11, slits penetrate the base member 4 from one surface to the other surface of the base member 4. The actual cut lines 11 are formed by penetration of the base member 4 with a blade. Note that the slits of the actual cut lines 11 may penetrate the bonding layer 6 or may not penetrate the bonding layer 6 depending on a manufacturing method (for example, in a case where the bonding layer 6 is formed on the base member 4 after penetration with a blade).

The cutting induction portion 10 includes the plurality of actual cut lines 11 having different shapes. In addition, the cutting induction portion 10 includes a pattern 13 formed of a combination of the plurality of actual cut lines 11 of different shapes. The cutting induction portion 10 includes the patterns 13 having identical shape arranged spacing apart from each other at a constant interval in the width direction D2.

In the present embodiment, the cutting induction portion 10 includes actual cut lines 11A and 11B having two different shapes. The actual cut line 11A and the actual cut line 11B are disposed alternately in the width direction D2. In addition, one pattern 13 is formed of a combination of the “actual cut line 11A and actual cut line 11B.” Note that while the “actual cut line 11A and actual cut line 11B” having an inverted V-like shape is regarded as the one pattern 13 for convenience of description here, the “actual cut line 11B and the actual cut line 11A” having a V-like shape may be regarded as the one pattern 13.

The actual cut line 11A slopes from the positive (one) side to the negative (the other) side of the width direction D2 toward the positive side (one side of the longitudinal direction) of the longitudinal direction D1. The actual cut line 11A includes an end 11Aa on the negative side of the width direction D2 and an end 11Ab on the positive side of the width direction D2. The actual cut line 11B slopes from the negative side to the positive side of the width direction D2 toward the positive side of the longitudinal direction D1. The actual cut line 11B includes an end 11Ba on the positive side of the width direction D2 and an end 11Bb on the negative side of the width direction D2. Both of the actual cut lines 11A and 11B slope straight in a straight line. Inclination angles θ of the actual cut lines 11A and 11B may be set in the range from 30 to 75°. The actual cut line 11A and the actual cut line 11B have a line symmetry relationship with respect to a center line extending in the longitudinal direction D1.

The end 11Aa of the actual cut line 11A and the end 11Ba of the actual cut line 11B are disposed at the same positions in the longitudinal direction D1 and are spaced apart from each other in the width direction D2. Note that a state where two ends are “disposed at the same positions in the longitudinal direction D1” means a state where when a virtual line extending parallel to the width direction D2 is set to pass through one end, the other end is also disposed on the virtual line. The end 11Ab of the actual cut line 11A and the end 11Bb of the actual cut line 11B are disposed at positions spaced apart from the ends 11Aa and 11Ba on the negative side of the longitudinal direction D1 to be disposed at the same positions in the longitudinal direction D1. The end 11Ab of the actual cut line 11A and the end 11Bb of the actual cut line 11B are disposed to be spaced apart from each other by a separation distance greater than a separation distance between the ends 11Aa and 11Ba.

The patterns 13 are disposed at the same positions in the longitudinal direction D1 and are disposed at a constant interval in the width direction D2. For example, when three patterns 13A, 13B and 13C illustrated in the drawing are described, the end 11Bb of the actual cut line 11B of the pattern 13A and the end 11Ab of the actual cut line 11A of the pattern 13B are disposed at the same positions in the longitudinal direction D1 and are disposed to be spaced apart from each other in the width direction D2. The end 11Bb of the actual cut line 11B of the pattern 13B and the end 11Ab of the actual cut line 11A of the pattern 13C are disposed at the same positions in the longitudinal direction D1 and are disposed to be spaced apart from each other in the width direction D2. A separation distance between the end 11Bb of the actual cut line 11B of the pattern 13A and the end 11Ab of the actual cut line 11A of the pattern 13B, and a separation distance between the end 11Bb of the actual cut line 11B of the pattern 13B and the end 11Ab of the actual cut line 11A of the pattern 13C are equal to each other. Note that while the above-described separation distance is set to be equal to the separation distance between the ends 11Aa and 11Ba in the present embodiment, the above-described separation distance may be larger or smaller than the separation distance between the ends 11Aa and 11Ba. The positional relationship of the patterns 13A, 13B and 13C also applies to other continuous three patterns.

The virtual cut line 12 is a line defining a cutting plan position at which cutting is preferentially performed when the cutting is performed at the cutting induction portion 10. While the base member 4 continuously extends at the virtual cut line 12, the base member 4 preferentially breaks along the virtual cut line 12 at the time of tearing. The virtual cut line 12 is set by connecting ends of the plurality of actual cut lines 11 together.

Here, setting of the virtual cut line 12 will be described. Here, of the three patterns 13A, 13B and 13C, the virtual cut line 12 extending from the actual cut line 11A of the pattern 13B will be described. Since the actual cut line 11A includes the two ends 11Aa and 11Ab, virtual cut lines 12A and 12B extend from the ends 11Aa and 11Ab, respectively. Note that since a relationship described below also applies to the virtual cut lines 12A and 12B for other actual cut lines 11, the description of the virtual cut lines 12A and 12B will be omitted.

The virtual cut line 12A extending from the end 11Aa (first end) of the actual cut line 11A (first actual cut line) of the pattern 13B is connected to an end closest to the end 11Aa of the actual cut line 11A of the pattern 13B among the ends of the actual cut lines 11 different from the actual cut line 11A of the pattern 13B. As the actual cut lines 11 different from the actual cut line 11A of the pattern 13B, there are the actual cut lines 11A and 11B of the pattern 13A, the actual cut line 11B of the pattern 13B, and the actual cut lines 11A and 11B of the pattern 13C. Of the ends of these actual cut lines 11, the end closest to the end 11Aa of the actual cut line 11A of the pattern 13B is the end 11Ba of the actual cut line 11B (second actual cut line) of the pattern 13B. Accordingly, the end 11Aa of the actual cut line 11A of the pattern 13B and the end 11Ba of the actual cut line 11B of the pattern 13B are connected together, and thus the virtual cut line 12A is set.

The virtual cut line 12B extending from the end 11Ab (second end) of the actual cut line 11A (first actual cut line) of the pattern 13B is connected to the end closest to the end 11Ab of the actual cut line 11A of the pattern 13B among the ends of the actual cut lines 11 different from the actual cut line 11A of the pattern 13B and the actual cut line 11B of the pattern 13B. There are the actual cut lines 11A and 11B of the pattern 13A and the actual cut lines 11A and 11B of the pattern 13C as the actual cut lines 11 different from the actual cut line 11A of the pattern 13B and the actual cut line 11B of the pattern 13B. Of the ends of these actual cut lines 11, the end closest to the end 11Ab of the actual cut line 11A of the pattern 13B is the end 11Bb of the actual cut line 11B (third actual cut line) of the pattern 13A. Accordingly, the end 11Ab of the actual cut line 11A of the pattern 13B and the end 11Bb of the actual cut line 11B of the pattern 13A are connected together, and thus the virtual cut line 12B is set.

In each cutting induction portion 10, a plurality of rows 14 each including the plurality of virtual cut lines 12 arranged in the width direction D2 are formed in the longitudinal direction D1. In the present embodiment, a plurality of the virtual cut lines 12A connecting the ends 11Aa and 11Ba are arranged in the width direction D2. Accordingly, a row 14A of the virtual cut lines 12A is formed. In addition, a plurality of the virtual cut lines 12B connecting the ends 11Ab and 11Bb are arranged in the width direction D2 at a position different from the virtual cut line 12A in the longitudinal direction D1. Accordingly, the row of the virtual cut lines 12B is formed. Therefore, two rows 14 of the virtual cut lines 12 are formed in each cutting induction portion 10.

The virtual cut line 12A extends in a straight line in the width direction D2 in parallel with the width direction D2. In the present embodiment, the plurality of virtual cut lines 12A are disposed at the same positions in the longitudinal direction D1. Between one virtual cut line 12A and another virtual cut line 12A, that is, between the ends 11Ba and 11Aa, the base layer 4 continuously extends in a region wider in the width direction D2 than at least the virtual cut line 12A. The virtual cut line 12B extends in a straight line in the width direction D2 in parallel with the width direction D2. In the present embodiment, the plurality of virtual cut lines 12B are disposed at the same positions in the longitudinal direction D1. Between one virtual cut line 12B and another virtual cut line 12B, that is, between the ends 11Ab and 11Bb, the base layer 4 continuously extends in a region wider in the width direction D2 than at least the virtual cut line 12B.

As illustrated in FIG. 4B, a projection reference line SL1 (first projection reference line) extending in the longitudinal direction D1 is set, and a plurality of projected portions 16 (first projected portions) are set by projecting the plurality of virtual cut lines 12 in each cutting induction portion 10 onto the projection reference line SL1. At this time, the projected portions 16 of the plurality of virtual cut lines 12 belonging to the same row 14 overlap each other. The projected portions 16 of the plurality of virtual cut lines 12 belonging to one row 12 and the projected portions 16 of the plurality of virtual cut lines 12 belonging to another row 12 are spaced apart from each other in the longitudinal direction D1 without overlapping each other.

Specifically, projected portions 16A are set by projecting the plurality of virtual cut lines 12A in each cutting induction portion 10 onto the projection reference line SL1. Projected portions 16B are set by projecting the plurality of virtual cut lines 12B in each cutting induction portion 10 onto the projection reference line SL1. The plurality of virtual cut lines 12A are parallel to the width direction D2, and are disposed at the same positions in the longitudinal direction D1. Therefore, the projected portions 16A of the plurality of virtual cut lines 12A belonging to the same row 14A overlap each other. Therefore, the plurality of projected portions 16A are set as one point on the projection reference line SL1. The plurality of virtual cut lines 12B are parallel to the width direction D2 and are disposed at the same positions in the longitudinal direction D1. Therefore, the projected portions 16B of the plurality of virtual cut lines 12B belonging to the same row 14B overlap each other. Therefore, the plurality of projected portions 16B are set as one point on the projection reference line SL1. The projected portions 16A of the plurality of virtual cut lines 12A belonging to the one row 14A and the projected portions 16B of the plurality of virtual cut lines 12B belonging to another row 14B are spaced apart from each other by a dimension L1 in the longitudinal direction D1 without overlapping each other.

A projection reference line SL2 (second projection reference line) extending in the width direction D2 is set, and a plurality of projected portions 17 (second projected portions) are set by projecting the plurality of virtual cut lines 12 in each cutting induction portion 10 onto the projection reference line SL2. At this time, the plurality of projected portions 17 are spaced apart in the width direction D2 without overlapping each other.

Specifically, the plurality of virtual cut lines 12A and 12B in each cutting induction portion 10 are projected onto the projection reference line SL2, and thus a plurality of projected portions 17A and 17B are alternately set. The virtual cut lines 12B are disposed on both sides in the width direction D2 of the virtual cut line 12A at positions spaced apart in the width direction D2 from the virtual cut line 12A. Therefore, the plurality of projected portions 17A and 17B are spaced apart by dimensions L2 and L3 in the width direction D2 without overlapping each other. The dimensions L2 and L3 are dimensions obtained when the actual cut lines 11A and 11B are projected onto the projection reference line SL2. In the present embodiment, the dimensions L2 and L3 are set to be equal.

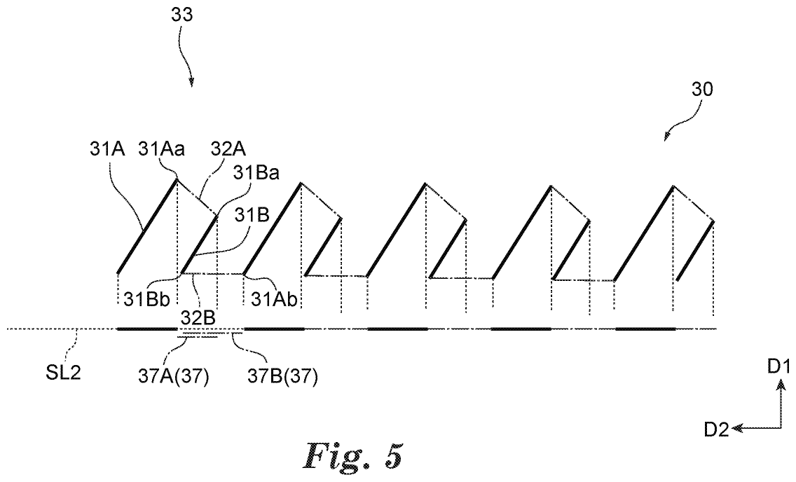

As a comparative example, a cutting induction portion 30 is illustrated in FIG. 5. The cutting induction portion 30 includes a pattern 33 including actual cut lines 31A and 31B inclined in the same direction. A virtual cut line 32A is set by connecting an end 31Aa of the actual cut line 31A and an end 31Ba of the actual cut line 31B. A virtual cut line 32B is set by connecting an end 31Bb of the actual cut line 31B and an end 31Ab of the actual cut line 31A of the pattern 13 adjacent. In this comparison example, when projected portions 37A and 37B of a plurality of the virtual cut lines 32A and 32B are set with respect to a projection reference line SL2, the projected portion 37A and the projected portion 37B partially overlap each other. Note that the projected portion 37A and the projected portion 37B completely overlap each other on the projection reference line SL2 as in locations that are not denoted by 37A and 37B. However, in FIG. 5, the projected portion 37A and the projected portion 37B are slightly spaced away from the projection reference line SL2 only at the locations denoted by “37A” and “37B” for the purpose of distinguishing the projected portion 37A from the projected portion 37B. Accordingly, the configuration according to this comparative example is not within the scope of an aspect of the present invention, and cannot provide the effect of the present invention.

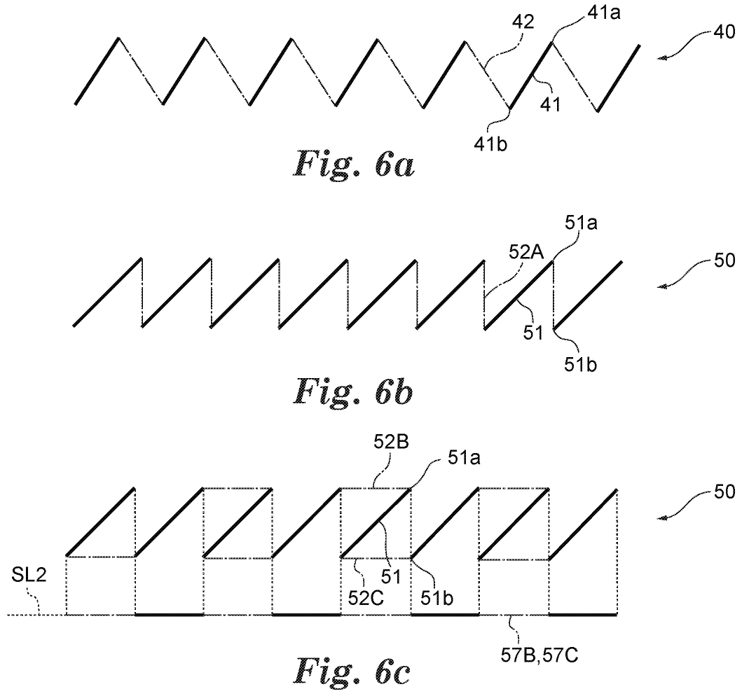

In addition, as a comparative example, a cutting induction portion 40 is illustrated in FIG. 6A. The cutting induction portion 40 includes actual cut lines 41 inclined and arranged in the width direction D2. With respect to such actual cut lines 41, a virtual cut line 42 is set by connecting an end 41a of one actual cut line 41 and an end 41b of another actual cut line 41. Only one row of virtual cut lines 42 of such a cutting induction portion 40 can be set. Accordingly, the configuration according to this comparative example is not within the scope of an aspect of the present invention and cannot provide the effect of the present invention.

In addition, as a comparative example, a cutting induction portion 50 is illustrated in FIG. 6B. The cutting induction portion 50 includes actual cut lines 51 inclined and arranged in the width direction D2. A separation distance between an end 51a of one actual cut line 51 and an end 51a of another actual cut line 51 is equal to a separation distance between the end 51a of the one actual cut line 51 and an end 51b of the other actual cut line 51. In this case, a virtual cut line 52A as illustrated in FIG. 6B and virtual cut lines 52B and 52C as illustrated in FIG. 6C can be set. However, when the virtual cut line 52A illustrated in FIG. 6B is set, only one row of the virtual cut lines can be set. In addition, when the virtual cut lines 52B and 52C as illustrated in FIG. 6C are set, projected portions 57B and 57C of the virtual cut lines 52B and 52C overlap each other and are not spaced apart in the width direction D2. Accordingly, the configuration according to this comparative example is not within the scope of an aspect of the present invention and cannot provide the effect of the present invention.

Note that, a relationship between the projected portions and the projection reference line SL1, and a relationship between the projected portions and the projection reference line SL2 described with reference to FIG. 4B are established in an entire region in the width direction D2 in the cutting induction portion 10 according to the present embodiment. However, the above-described relationships may not be established in a small region in the width direction D2 as long as the effect of the cutting induction portion 10 is not inhibited. Conversely, when the above-described relationships are established only in a small region in the width direction D2, the cutting induction portion cannot provide the effect of the cutting induction portion 10 of the present application and does not serve as the “cutting induction portion” of the present application. For example, the above-described relationships may be established in 50, 70, or 80% or greater of an entire region in the width direction D2. In addition, in the present embodiment, the above-described relationships are established in all of the plurality of cutting induction portions 10 provided to be spaced apart from each other in the longitudinal direction D1. However, some of the cutting induction portions in the longitudinal direction D1 of the tape 2 may not satisfy the above-described relationships.

Next, operations and effects of the roll product 1 according to the present embodiment will be described. Note that the following describes the operations and effects merely from one side and is not intended to exclude the case where the effects of the invention of the present application can be achieved by other factors.

In the roll product 1 according to the present embodiment, the base material 4 and the plurality of cutting induction portions 10 extending in the width direction D2 of the tape 2 are formed in the longitudinal direction D1 of the tape 2. The cutting induction portions 10 are arranged to be spaced apart from each other in the width direction D2 and each include a combination of the plurality of actual cut lines 11 formed by partially cutting the base member 4, and the plurality of virtual cut lines 12 set by connecting the ends of the plurality of actual cut lines 11. Here, in the plurality of actual cut lines 11, the virtual cut line 12A extending from the end 11Aa (first end) of the actual cut line 11A (first actual cut line) of the pattern 13B is connected to an end closest to the end 11Aa of the actual cut line 11A of the pattern 13B among the ends of the actual cut lines 11 different from the actual cut line 11A of the pattern 13B. Here, the virtual cut line 12A is set by connecting the end 11Aa of the actual cut line 11A of the pattern 13B and the end 11Ba of the actual cut line 11B of the pattern 13B. In addition, the virtual cut line 12B extending from the end 11Ab (second end) of the actual cut line 11A (first actual cut line) of the pattern 13B is connected to an end closest to the end 11Ab of the actual cut line 11A of the pattern 13B among the ends of the actual cut lines 11 different from the actual cut line 11A of the pattern 13B and the actual cut line 11B of the pattern 13B. Here, the virtual cut line 12B is set by connecting the end 11Ab of the actual cut line 11A of the pattern 13B and the end 11Bb of the actual cut line 11B of the pattern 13A. Thus, since the virtual cut lines 12 are set by connecting adjacent ends of the actual cut lines 11, cutting is preferentially performed at positions of the virtual cut lines 12 at the time of tearing of the tape 2. In the cutting induction portion 10, since such virtual cut lines 12 and such actual cut lines 11 are alternatively and continuously arranged in the width direction D2, the tape 2 is easily torn at the position of the cutting induction portion 10.

Here, the row 14 of the virtual cut lines 12 formed in each cutting induction portion 10 will be described. In a case where the projection reference line SL1 extending in the longitudinal direction D1 is set, and the plurality of projected portions 16 are set by projecting the plurality of virtual cut lines 12 in each cutting induction portion 10 onto the projection reference line SL1, the projected portions 16 of the plurality of virtual cut lines 12 belonging to the same row 14 overlap each other. Thus, since locations corresponding to the rows 14 of the virtual cut lines 12 are locations where the plurality of virtual cut lines 12 are densely provided in the longitudinal direction D1, tensile strength of the tape 2 is easily reduced at the locations. For example, in the comparative example illustrated in FIG. 6A, only one row of the virtual cut lines 42 is formed in each cutting induction portion 40. Therefore, when a tensile load in the longitudinal direction D1 is applied to the tape 2, the tensile load is concentrated at the location of the row of the virtual cut lines 42 arranged in a line, and the tape 2 is easily broken at the location. Thus, the configuration of the comparative example may not provide sufficient tensile strength. In contrast, in the roll product 1 according to the present embodiment, a plurality of the rows 14 of the virtual cut lines 12 arranged in the width direction D2 are formed in the longitudinal direction D1 in each cutting induction portion 10. In addition, the projected portions 16A of the plurality of virtual cut lines 12A belonging to one row 14A, and the projected portions 16B of the plurality of virtual cut lines 12B belonging to another row 14B are spaced apart in the longitudinal direction D1 without overlapping each other. Thus, when the rows 14A and 14B are disposed at locations spaced apart in the longitudinal direction D1 as described above, concentration of the tensile load can be distributed in the longitudinal direction D1. Therefore, tensile strength of the tape 2 can be improved.

Next, a configuration in which the projection reference line SL2 extending in the width direction D2 is set, and the plurality of projected portions 17 are set by projecting the plurality of virtual cut lines 12 in each cutting induction portion 10 onto the projection reference line SL2 will be described. First, as a comparative example, a configuration in which the projected portion 37A and the projected portion 37B projected on a projection reference line SL2 overlap as illustrated in FIG. 5, will be considered. In such a cutting induction portion 30, when tearing is performed in a direction from the negative side to the positive side of the width direction D2, the virtual cut line 32B is cut from the end 31Ab toward the end 31Bb, and then cutting is performed at the virtual cut line 32A via the actual cut line 31B. However, the end 31Ba at which cutting is started in the virtual cut line 32A is located upstream in the tearing direction (negative side of the width direction D2) of the end 31Bb of the virtual cut line 32B in which the cutting has already been performed. In such a positional relationship, the tearing of the tape 2 may not proceed smoothly along the width direction D2. Thus, in the configuration in which the projected portions projected on the projection reference line SL2 overlap, smooth tearing of the tape 2 may be inhibited, and hand-tearability may be reduced. In contrast, in the roll product 1 according to the present embodiment, the plurality of projected portions 17 are spaced apart in the width direction D2 without overlapping each other. In such a configuration, in a case where tearing is performed in the cutting induction portion 10, when cutting at one virtual cut line 12 is completed, next cutting in the virtual cut line 12 is started at a position downstream in the tearing direction of at least the virtual cut line 12 in which the cutting has already been performed. Specifically, in a case where tearing is performed from the negative side toward the positive side of the width direction D2 in the cutting induction portion 10, cutting is performed in the virtual cut line 12B from the end 11Ab toward the end 11Bb, and then cutting is performed in the virtual cut line 12A via the actual cut line 11B. The end 11Ba where cutting is started in the virtual cut line 12A is located downstream in the tearing direction (positive side of the width direction D2) of the end 11Bb of the virtual cut line 12B in which the cutting has already been performed. In such a positional relationship, the tearing of the tape 2 proceeds smoothly along the width direction D2. Accordingly, the roll product 1 according to the present embodiment can improve tensile strength of the tape 2 and can improve hand-tearability of the tape 2.

Two rows of the virtual cut lines 12 are formed in each cutting induction portion 10.

The actual cut line 11A inclined from one side to the other side of the width direction D2 toward one side of the longitudinal direction D1 and the actual cut line 11B inclined from the other side to one side of the width direction D2 toward one side of the longitudinal direction D1 are alternately arranged in the width direction D2.

The virtual cut line 12 extends in parallel with the width direction D2. In this case, at the time of tearing of the tape 2, the tape 2 is cut smooth and straight in the width direction D2 in the virtual cut line 12.

The present invention is not limited to the above-described embodiment.

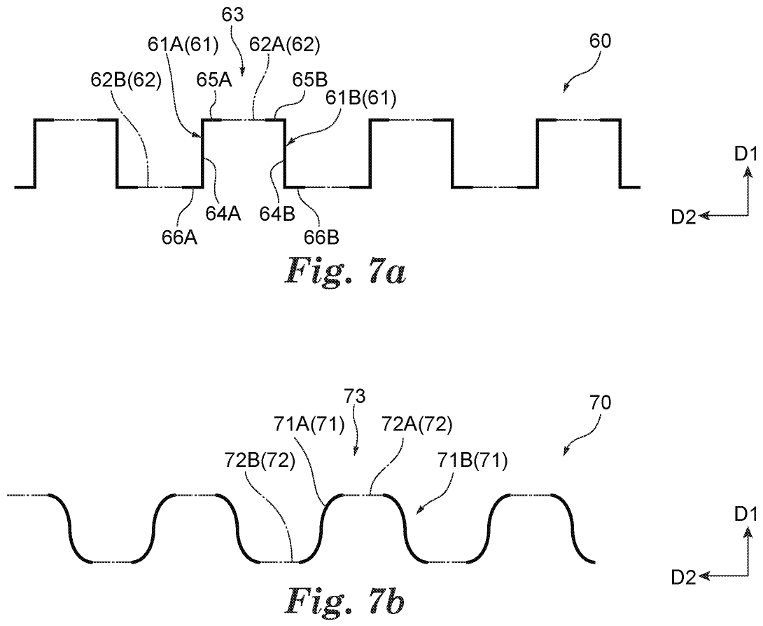

For example, the shape of the actual cut line is not limited to a shape inclined in a straight line. As illustrated in FIG. 7A, a cutting induction portion 60 including an actual cut line 61 bent may be employed. The cutting induction portion 60 includes a pattern 63 of actual cut lines 61A and 61B. The actual cut line 61A includes a straight portion 64A extending straight in the longitudinal direction D1, a bent portion 65A extending from an end on the positive side of the longitudinal direction D1 of the linear portion 64A to the negative side of the width direction D2, and a bent portion 66A extending from an end on the negative side of the longitudinal direction D1 of the linear portion 64A to the positive side of the width direction D2. The actual cut line 61B includes a straight portion 64B extending straight in the longitudinal direction D1, a bent portion 65B extending from an end on the positive side of the longitudinal direction D1 of the linear portion 64B to a positive side of the width direction D2, and a bent portion 66B extending from an end on the negative side of the longitudinal direction D1 of the linear portion 64B to the negative side of the width direction D2. Note that the cutting induction portion 60 includes virtual cut lines 62A and 62B similar to the virtual cut lines 12A and 12B of the cutting induction portion 10.

In addition, as illustrated in FIG. 7B, a cutting induction portion 70 including an actual cut line 71 curved may be employed. The cutting induction portion 70 includes a pattern 73 of actual cut lines 71A and 71B. The actual cut line 71A has a substantially S-shape obtained by curving a corner between the straight portion 64A and the bent portion 65A and curving a corner between the straight portion 64A and the bent portion 66A of the actual cut line 61A in FIG. 7A. The actual cut line 71B has a substantially inverted S-shape obtained by curving a corner between the straight portion 64B and the bent portion 65B and curving a corner between the straight portion 64B and the bent portion 66B of the actual cut line 61B in FIG. 7A. Note that the cutting induction portion 70 includes virtual cut lines 72A and 72B similar to the virtual cut lines 12A and 12B of the cutting induction portion 10.

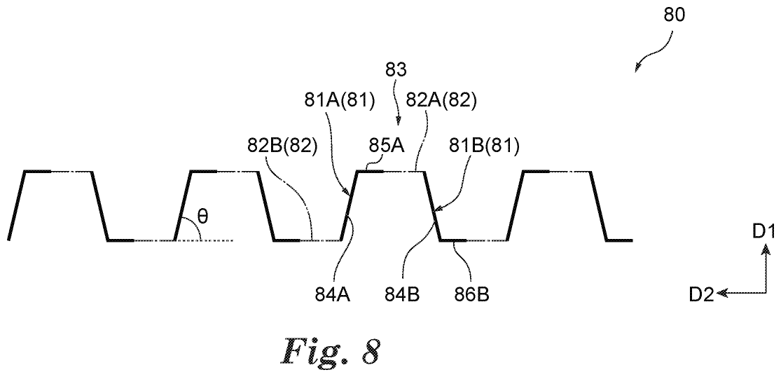

In the cutting induction portions 10, 60 and 70 illustrated in FIGS. 4A and 4B and 7A and 7B, a pair of actual cut lines in one pattern have a line symmetry relationship with each other, but the pair of actual cut lines in one pattern may not have a line symmetry relationship. For example, a cutting induction portion 80 as illustrated in FIG. 8 may be employed. The cutting induction portion 80 includes a pattern 83 of actual cut lines 81A and 81B. The actual cut line 81A includes a slope 84A inclined in a straight line, and a bent portion 85A extending from an end on the positive side of the longitudinal direction D1 of the slope 84A to the negative side of the width direction D2. The actual cut line 81B includes a slope 84B inclined in a straight line in the longitudinal direction D1, and a bent portion 86B extending from an end on the negative side of the longitudinal direction D1 of the slope 84B to the negative side of the width direction D2. Note that a virtual cut line 82A of the cutting induction portion 80 is set by connecting a tip end of the bent portion 85A of the actual cut line 81A and an end on the positive side of the longitudinal direction D1 of the slope 84B of the actual cut line 81B. A virtual cut line 82B of the cutting induction portion 80 is set by connecting a tip end of the bent portion 86B of the actual cut line 81B and an end on the negative side of the longitudinal direction D1 of the slope 84A of the actual cut line 81A.

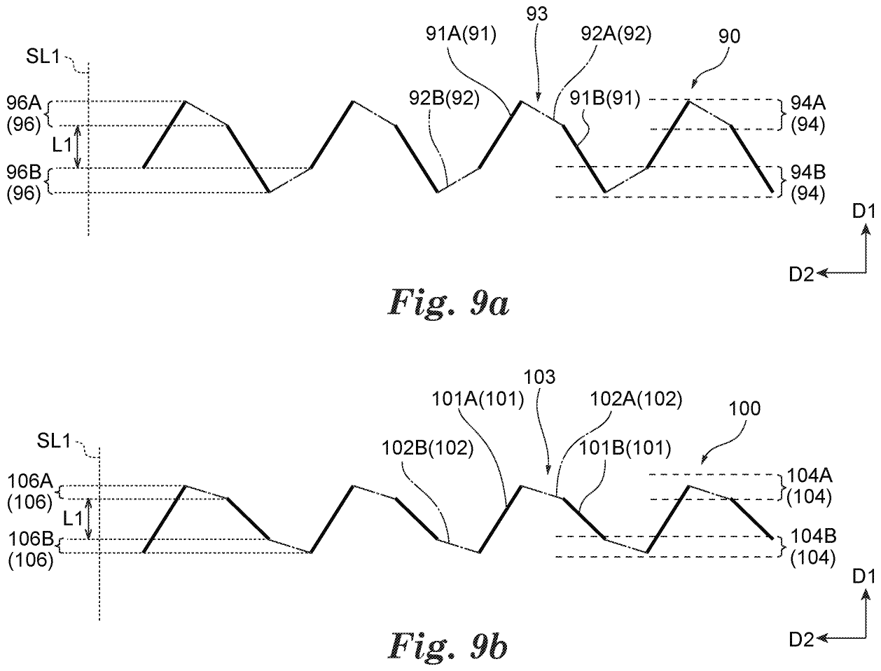

In the cutting induction portions 10, 60, 70, and 80 illustrated in FIGS. 4A and 4B, 7A and 7B, and 8, the positions in the longitudinal direction D1 of the both ends of the pair of actual cut lines in one pattern coincide with each other, but the positions may not coincide with each other. For example, a cutting induction portion 90 as illustrated in FIG. 9A may be employed. In the cutting induction portion 90, in a pattern 93, an actual cut line 91A and an actual cut line 91B are disposed at positions shifted from each other in the longitudinal direction D1. Specifically, the actual cut line 91A includes a configuration in which the actual cut line 11A illustrated in FIGS. 4A and 4B is moved to the positive side of the longitudinal direction D1, and the actual cut line 91B includes a configuration in which the actual cut line 11B illustrated in FIGS. 4A and 4B is moved to the negative side of the longitudinal direction D1. In this case, a virtual cut line 92A is inclined toward the negative side of the longitudinal direction D1 in a direction from the positive side toward the negative side of the width direction D2 between the actual cut line 91A and the actual cut line 91B. In addition, a virtual cut line 92B is inclined toward the positive side of the longitudinal direction D1 in a direction from the positive side to the negative side of the width direction D2 between the actual cut line 91B and the actual cut line 91A.

Projected portions 96A are set by projecting a plurality of the virtual cut lines 92A in each cutting induction portion 90 on a projection reference line SL1. Projected portions 96B are set by projecting a plurality of the virtual cut lines 92B in each cutting induction portion 90 on the projection reference line SL1. The plurality of virtual cut lines 92A are disposed at the same positions in the longitudinal direction D1 in a state where the plurality of virtual cut lines 92A are inclined with respect to the width direction D2. Therefore, the projected portions 96A of the plurality of virtual cut lines 92A belonging to the same row 94A overlap each other. The plurality of projected portions 96A are set as line segments each having a fixed length along the longitudinal direction D1 on the projection reference line SL1. The plurality of virtual cut lines 92B are disposed at the same positions in the longitudinal direction D1 in a state where the plurality of virtual cut lines 92B are inclined with respect to the width direction D2. Therefore, the projected portions 96B of the plurality of virtual cut lines 92B belonging to the same row 94B overlap each other. Therefore, regarding the plurality of projected portions 96B, the projected portions 96A of the plurality of virtual cut lines 92A belonging to the one row 94A set as a line segment having a fixed length along the longitudinal direction D1 on the projection reference line SL1, and the projected portions 96B of the plurality of virtual cut lines 92B belonging to the other row 94B are spaced apart by a dimension L1 in the longitudinal direction D1 without overlapping each other.

In the cutting induction portions 10, 60, 70, 80, and 90 illustrated in FIGS. 4A and 4B, 7A and 7B, 8, and 9A, the lengths in the longitudinal direction D1 of the pair of actual cut lines in one pattern are identical to each other, but the lengths may not be identical to each other. For example, a cutting induction portion 100 as illustrated in FIG. 9B may be employed. In the cutting induction portion 100, an actual cut line 101A and an actual cut line 101B in a pattern 103 have different lengths in the longitudinal direction D1. Specifically, the actual cut line 91B includes a configuration in which a distance between ends on the positive side and the negative side of the longitudinal direction D1 of the actual cut line 11B illustrated in FIGS. 4A and 4B is shortened. In this case, a virtual cut line 102A is inclined toward the negative side of the longitudinal direction D1 in a direction from the positive side toward the negative side of the width direction D2 between the actual cut line 101A and the actual cut line 101B. In addition, a virtual cut line 102B is inclined toward the negative side of the longitudinal direction D1 in a direction from the positive side toward the negative side of the width direction D2 between the actual cut line 101B and the actual cut line 101A. Projected portions 106A of a plurality of the virtual cut lines 102A belonging to the same row 104A overlap each other. The plurality of projected portions 106A are set as line segments each having a fixed length along the longitudinal direction D1 on a projection reference line SL1. Projected portions 106B of a plurality of the virtual cut lines 102B belonging to the same row 104B overlap each other. The plurality of projected portions 106B are set as line segments each having a fixed length along the longitudinal direction D1 on the projection reference line SL1.

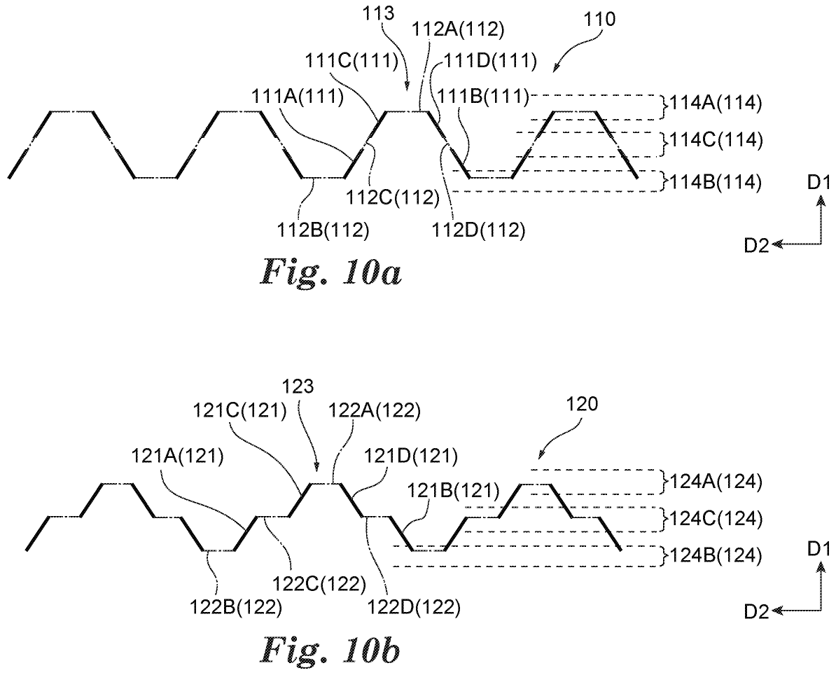

In the cutting induction portions 10, 60, 70, 80, 90, and 100 illustrated in FIGS. 4A and 4B, 7A and 7B, 8, and 9A and 8B, one pattern includes a pair of actual cut lines. Alternatively, one pattern may include three or more actual cut lines. For example, a cutting induction portion 110 as illustrated in FIG. 10A may be employed. The cutting induction portion 110 includes a pattern 113 including four actual cut lines 111A, 111B, 111C and 111D. The actual cut lines 111A and 111C include a configuration in which an uncut portion is formed at a center position in the longitudinal direction D1 in the actual cut line 11A illustrated in FIGS. 4A and 4B. The actual cut lines 111B and 111D include a configuration in which an uncut portion is formed at a center position in the longitudinal direction D1 in the actual cut line 11B illustrated in FIGS. 4A and 4B. In this case, ends on the positive side of the longitudinal direction D1 of the actual cut line 111C and the actual cut line 111D are connected, and thus a virtual cut line 112A similar to the virtual cut line 12A illustrated in FIGS. 4A and 4B is set. Ends on the negative side of the longitudinal direction D1 of the actual cut line 111A and the actual cut line 111B are connected, and thus a virtual cut line 112B similar to the virtual cut line 12B illustrated in FIGS. 4A and 4B is set. An end on the positive side of the longitudinal direction D1 of the actual cut line 111A and an end on the negative side of the longitudinal direction D1 of the actual cut line 111C are connected, and thus a virtual cut line 112C is set. A combination of the actual cut lines 111A and 111C and the virtual cut line 112C forms one inclined straight line. An end on the positive side of the longitudinal direction D1 of the actual cut line 111B and an end on the negative side of the longitudinal direction D1 of the actual cut line 111D are connected, and thus a virtual cut line 112D is set. A combination of the actual cut lines 111B and 111D and the virtual cut line 112D forms one inclined straight line. Since the virtual cut line 112C and the virtual cut line 112D are formed at the same positions in the longitudinal direction D1, the virtual cut line 112C and the virtual cut line 112D belong to the same row 114C. Accordingly, the cutting induction portion 110 includes rows 114A, 114B and 114C as rows of virtual cut lines 112.

In addition, a cutting induction portion 120 as illustrated in FIG. 10B may be employed. The cutting induction portion 120 includes a pattern 123 including four actual cut lines 121A, 121B, 121C and 121D. The cutting induction portion 120 differs from the cutting induction portion of FIG. 10A in that virtual cut lines 122C and 122D belonging to a central row 124C are parallel to the width direction D2. Therefore, the actual cut lines 121A and 121C include a configuration in which the actual cut lines 111A and 111C in FIG. 10A are slid to be spaced apart from each other in the width direction D2 and are moved closer to each other in the longitudinal direction D1. The actual cut lines 121B and 121D include a configuration in which the actual cut lines 111B and 111D in FIG. 10A are spaced apart from each other in the width direction D2 and are moved closer to each other in the longitudinal direction D1. The cutting induction portion 120 includes rows 124A, 124B and 124C as rows of the virtual cut lines 122.

Note that in the cutting induction portion, the pattern of the actual cut lines may not be the same across an entire region in the width direction D2. For example, in the cutting induction portion, any of the above-described patterns may be employed in a certain region in the width direction D2, and other patterns may be employed in other certain regions. In addition, different patterns may be formed alternately, or different patterns may be formed at a predetermined frequency (a different pattern is formed at a certain ratio).

### EXAMPLES

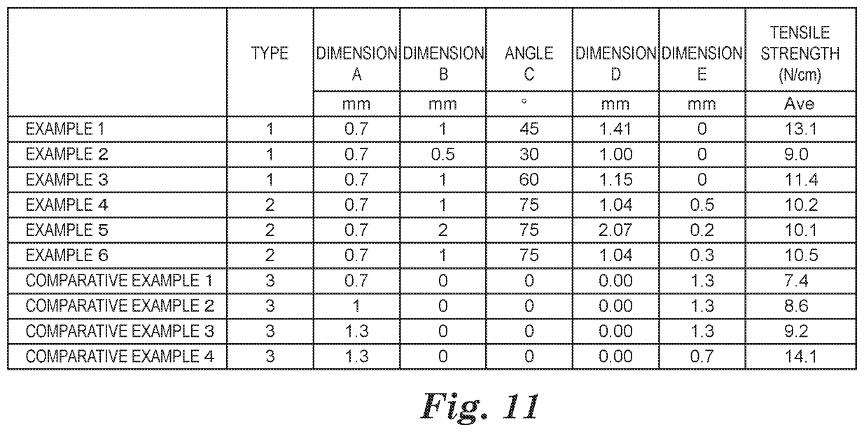

Examples will be described below, but the present invention is not limited to the examples. A tape described below was prepared in Examples 1 to 6 and Comparative Examples 1 to 4. As a medical nonwoven tape, a 25 mm-wide MICROPORE (trade name) surgical tape available from 3M Company was prepared. The tape was cut into dimensions in accordance with each experiment, and a cutting induction portion was formed in a measurement location. The conditions of the cutting induction portions in Examples 1 to 6 and Comparative Examples 1 to 4 are shown in the table in FIG. 11. Regarding the “type” in this table, the pattern of the type illustrated in FIGS. 4A and 4B was employed for “1,” the pattern of the type illustrated in FIG. 8 was employed for “2,” and a perforation was employed for “3.” The perforation is a cutting induction portion including actual cut lines arranged at an even pitch in parallel with the width direction. In addition, the “dimension A” is a length of the virtual cut line, and the “dimension B” is a value of a gap between the two rows of virtual cut lines (represented by “L1” in FIG. 4B). The “angle C” is a value of an inclination angle of the actual cut line with respect to the width direction (represented by “0” in FIGS. 4A and 8). The “dimension D” is a length of the slope (the entirety of the actual cut lines 11A and 11B in FIGS. 4A and 4B, and the slopes 84A and 84B in FIG. 8) of the actual cut line, and the “dimension E” is a length of the straight line portion (the bent portion 85A in a straight line and the bent portion 86 in a straight line in FIG. 8) of the actual cut line.

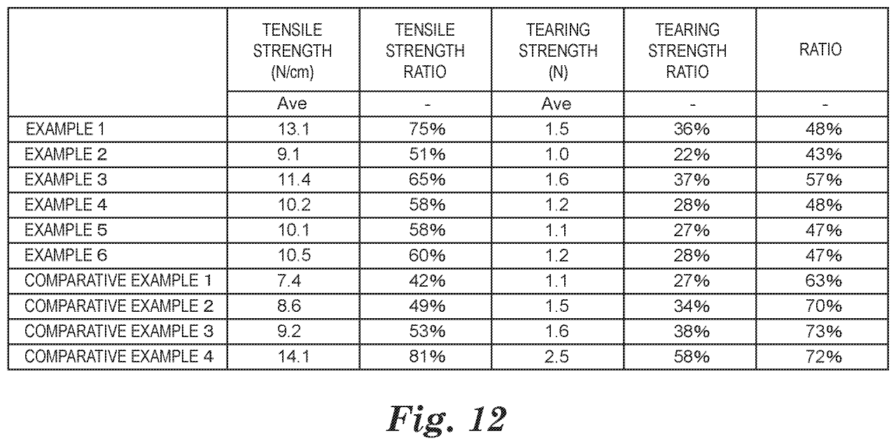

Tensile tests were performed in Examples 1 to 6 and Comparative Examples 1 to 4. In the tensile tests, a test piece having a width of 10 mm and a length of 100 mm was cut out of the tape. A cutting induction portion was formed at a center portion in the longitudinal direction of the test piece. The test piece was pinched with a pair of jigs spaced apart from each other at an interval of 50 mm, and the jigs were spaced apart from each other at a speed of 1000 mm/min to cause breakage, and strength at the time of breakage was determined as tensile strength. The tensile strengths (N/cm) thus determined are shown in the tables in FIGS. 11 and 12. In addition, the table of FIG. 12 shows the “tensile strength ratio” as a ratio of the tensile strength of Examples 1 to 6 and Comparative Examples 1 to 4 in a case where the tensile strength of a test piece in which no cutting induction portion is formed is set to 100%.

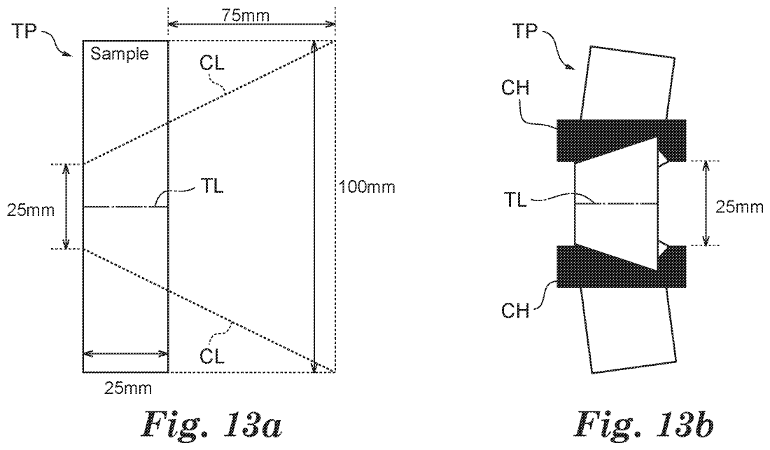

In addition, a tearing test was performed by a trapezoid method in Examples 1 to 6 and Comparative Examples 1 to 4. In the tearing test, a test piece having a width of 25 mm and a length of 100 mm was cut out. A cutting induction portion TL was formed at a center position in the longitudinal direction of the test piece. As illustrated in FIGS. 13A and 13B, lines CL are set at positions of sides of a trapezoid having an upper side of 25 mm, a bottom side of 100 mm, and a height of 75 mm in a test piece TP. The test piece TP was pinched with a pair of jigs CH along the lines CL, and the pair of jigs CH were spaced apart from each other in parallel at an interval of 25 mm. Then, the jigs CH were spaced apart from each other at a speed of 1000 mm/min to cause breakage, and average strength at the time of breakage was determined as tearing strength. The tearing strength (N) thus determined is shown in the table in FIG. 12. In addition, the table of FIG. 12 also shows the “tearing strength ratio” as a ratio of the tearing strength of Examples 1 to 6 and Comparative Examples 1 to 4 in a case where the tearing strength of the test piece in which no cutting induction portion is formed is set to 100%. In addition, a proportion of the “tearing strength ratio” to the “tensile strength ratio” is also shown in the item “ratio.”

### DRAWINGS IN EACH FORM

The following discloses a “front view,” a “rear view,” a “plan view,” a “bottom view,” a “right side view,” a “left side view,” a “partially enlarged view,” an “enlarged cross-sectional view,” a “reference partially enlarged perspective view showing each component name” and a “reference view illustrating a cut state” in each form. Note that in some drawings, features are indicated by solid lines, and other portions are indicated by dashed lines. In addition, the “plan view,” the “bottom view,” the “right side view” and the “left side view” share commonalities in all forms, and therefore will be omitted as appropriate.

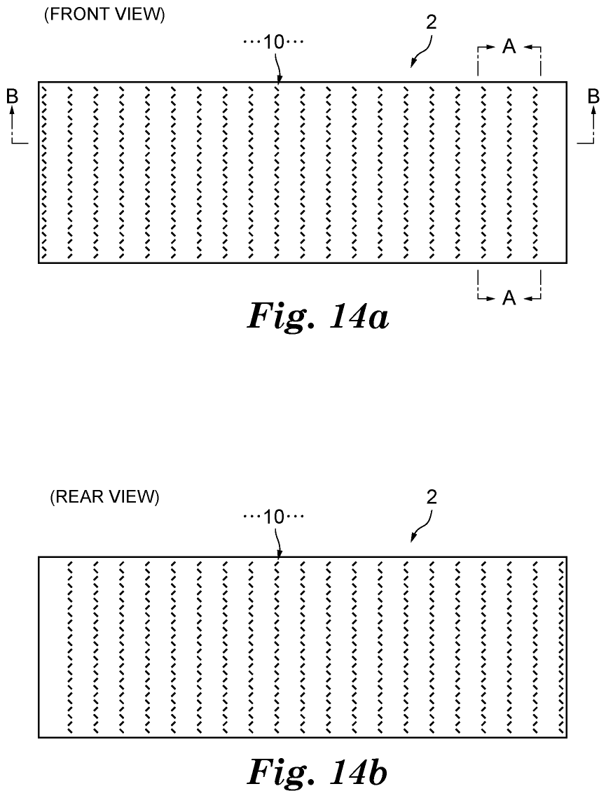

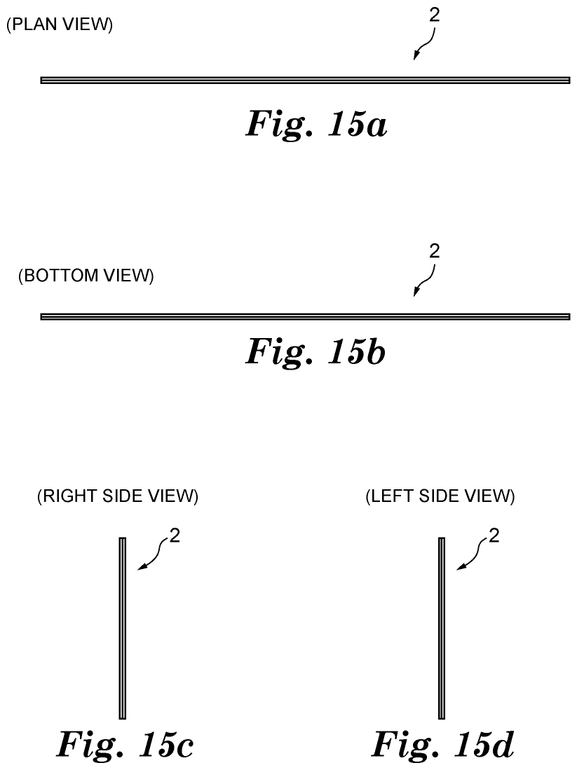

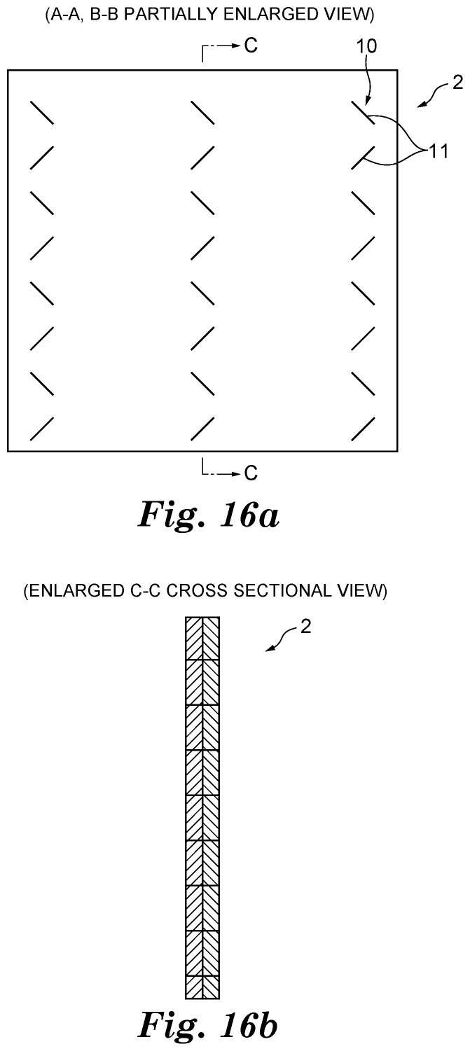

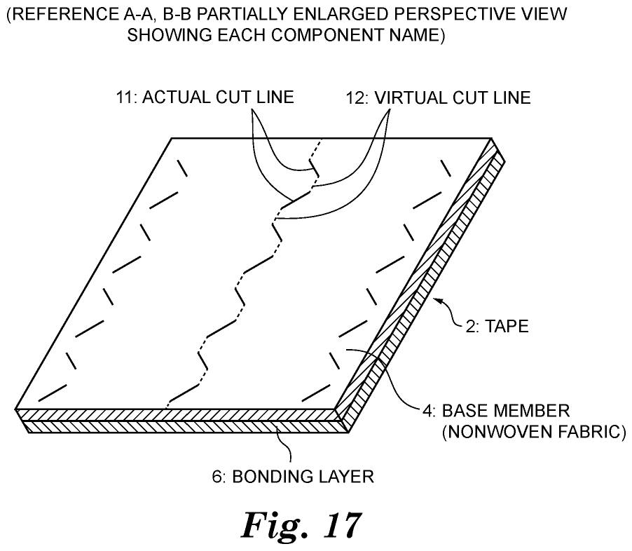

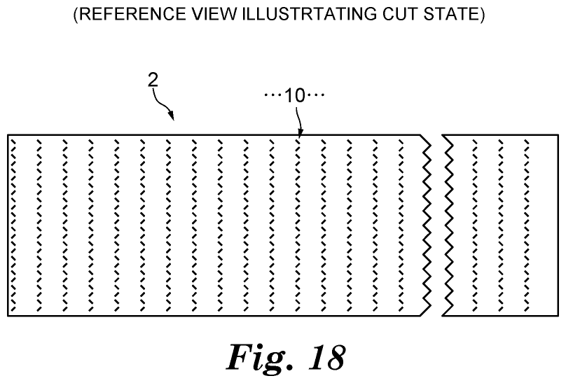

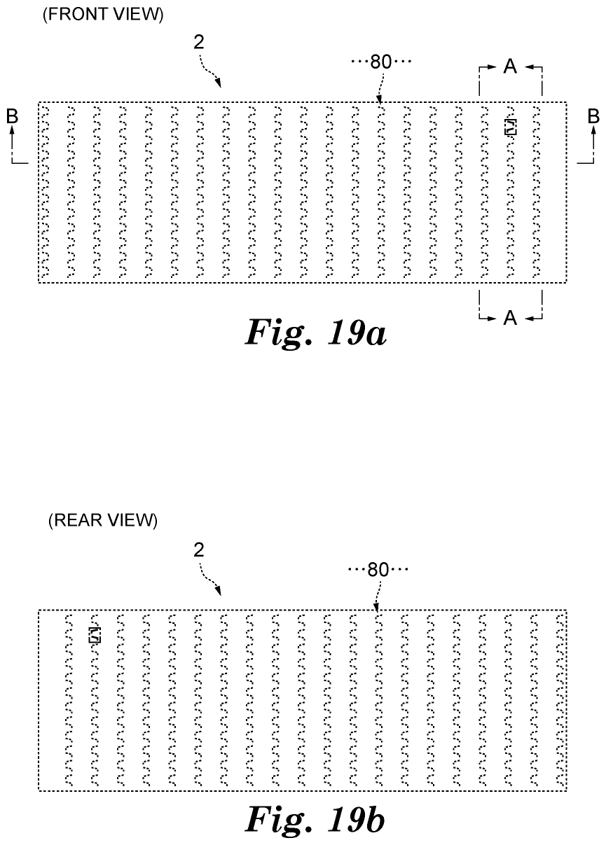

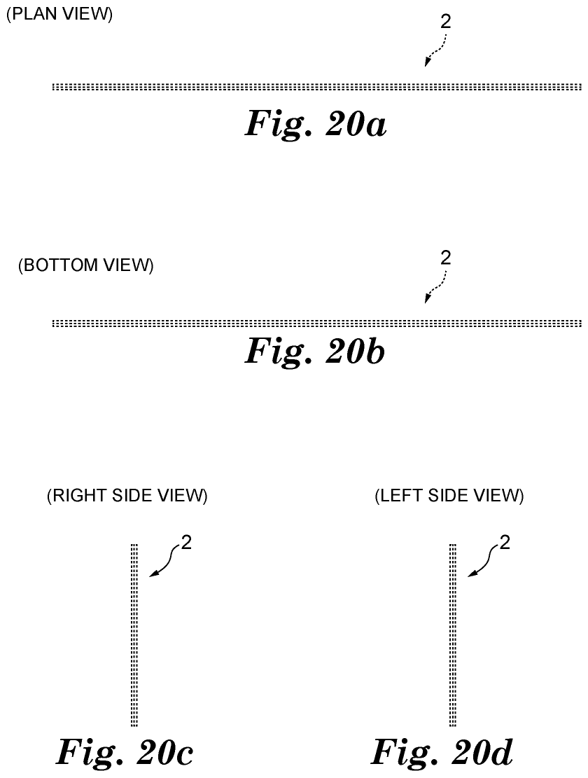

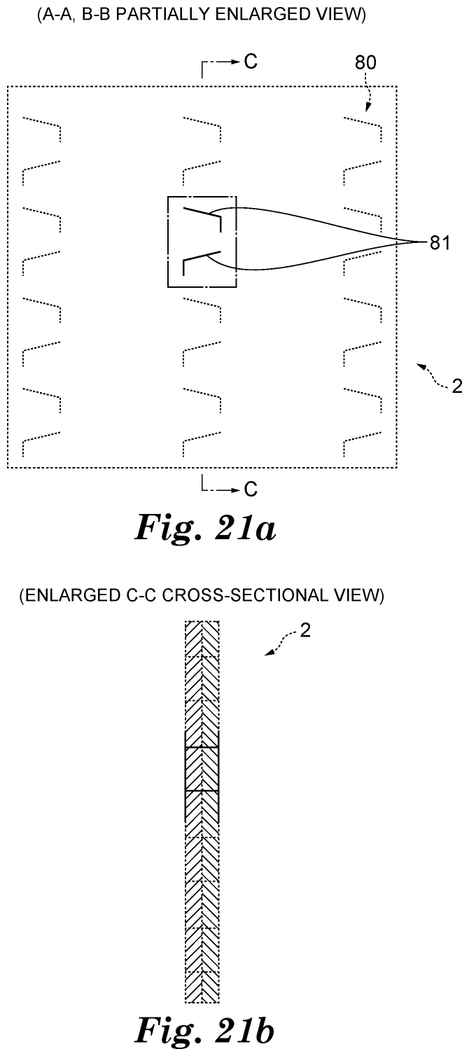

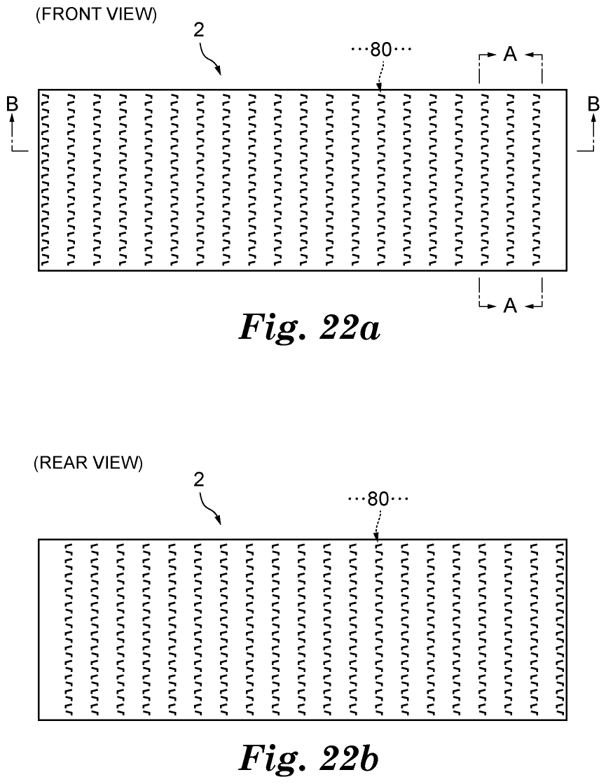

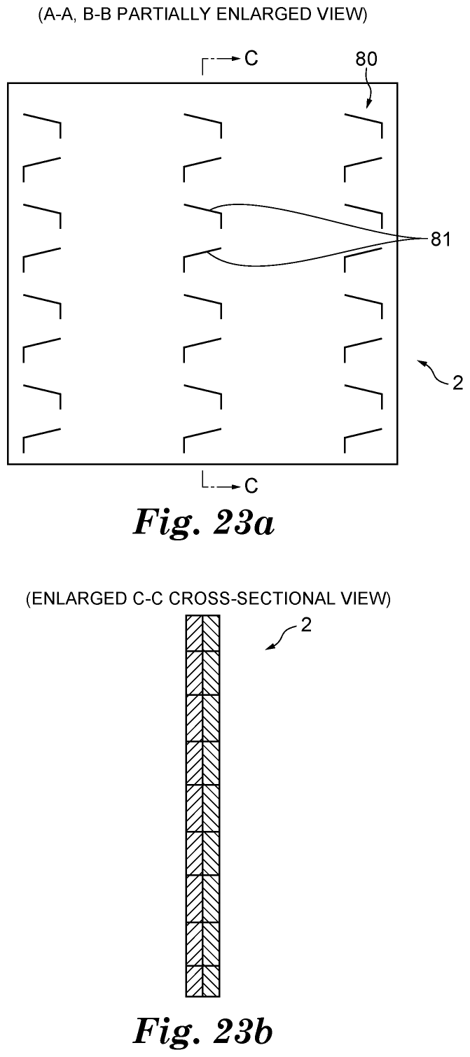

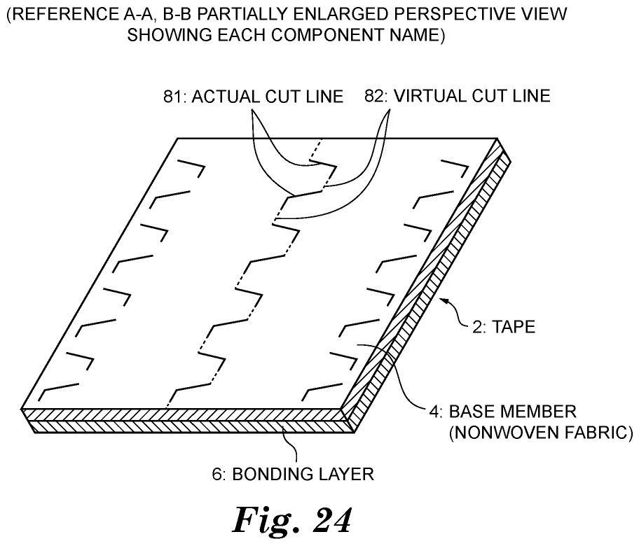

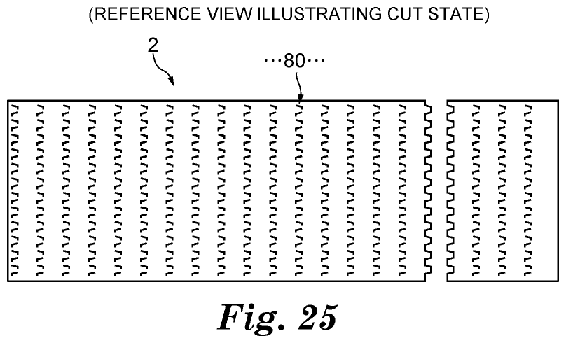

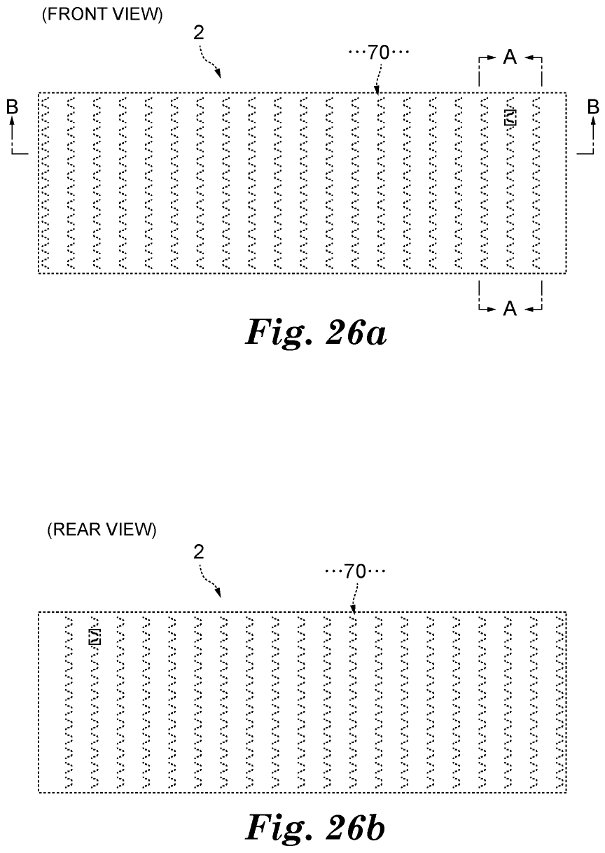

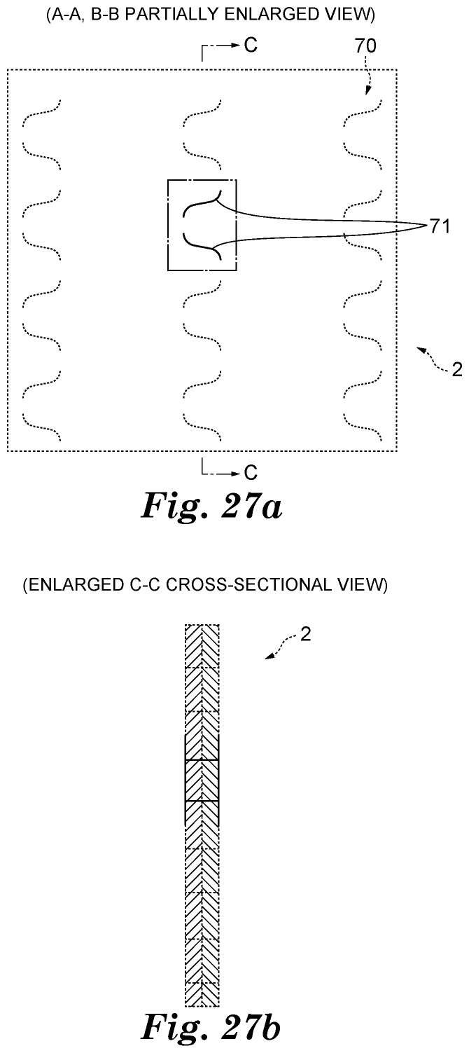

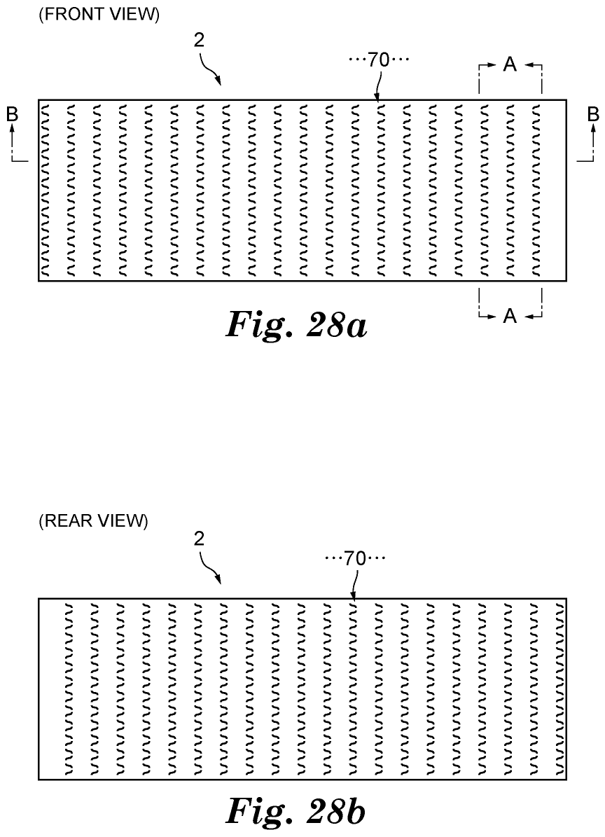

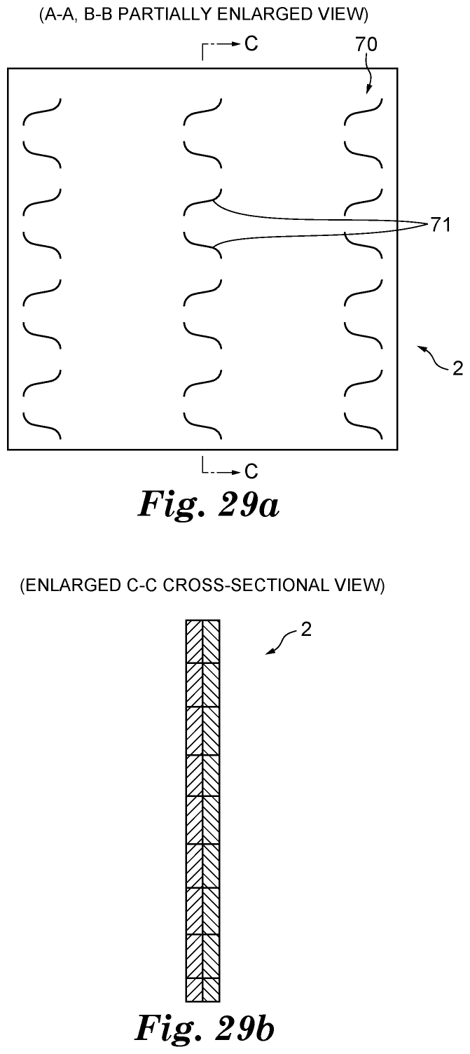

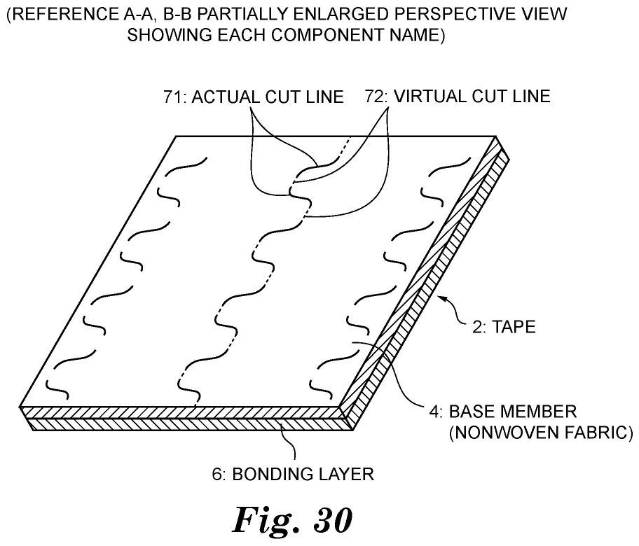

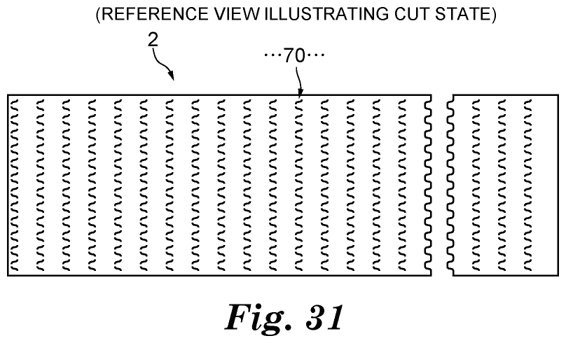

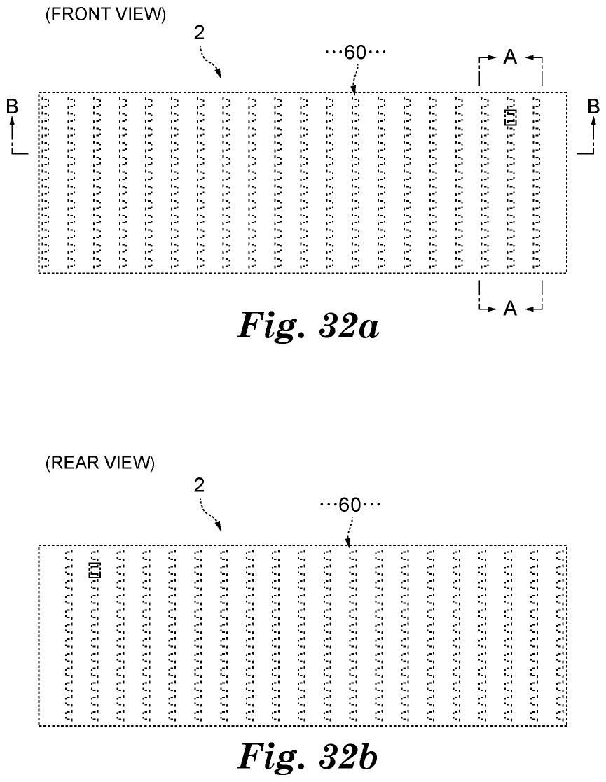

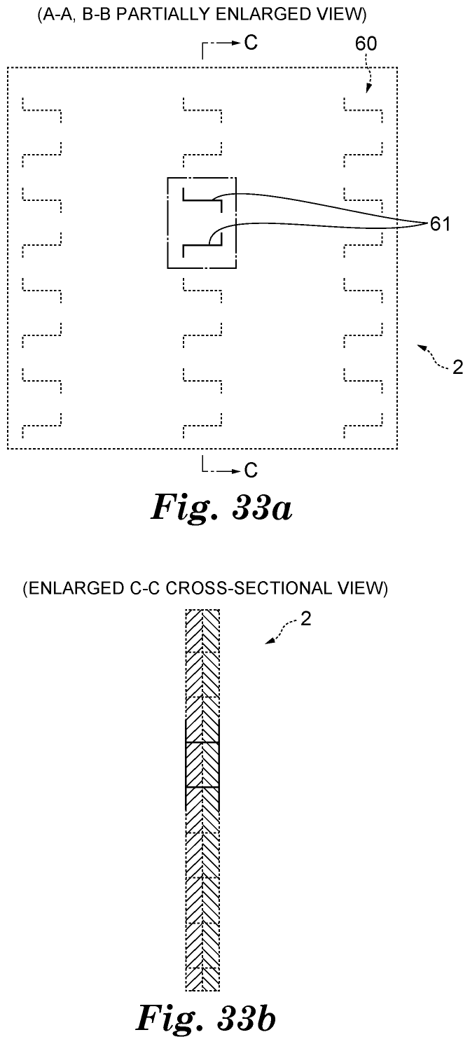

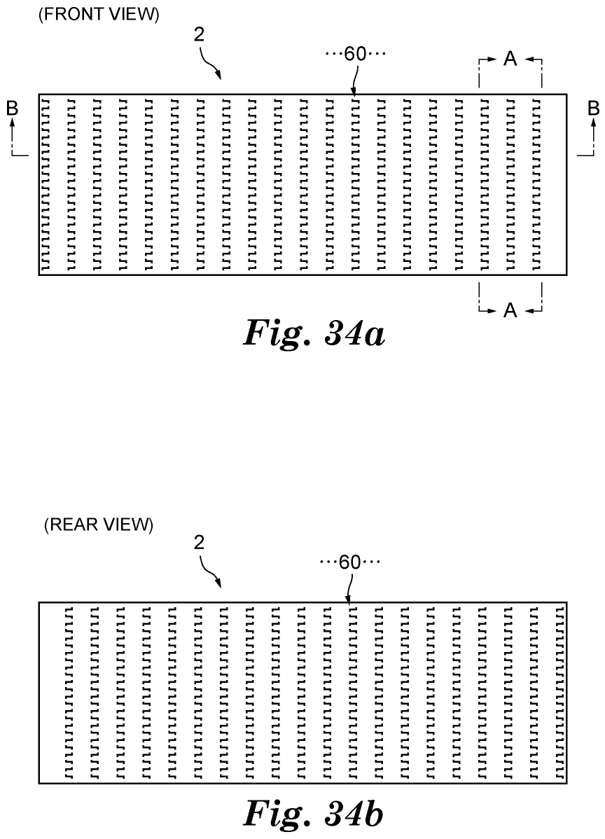

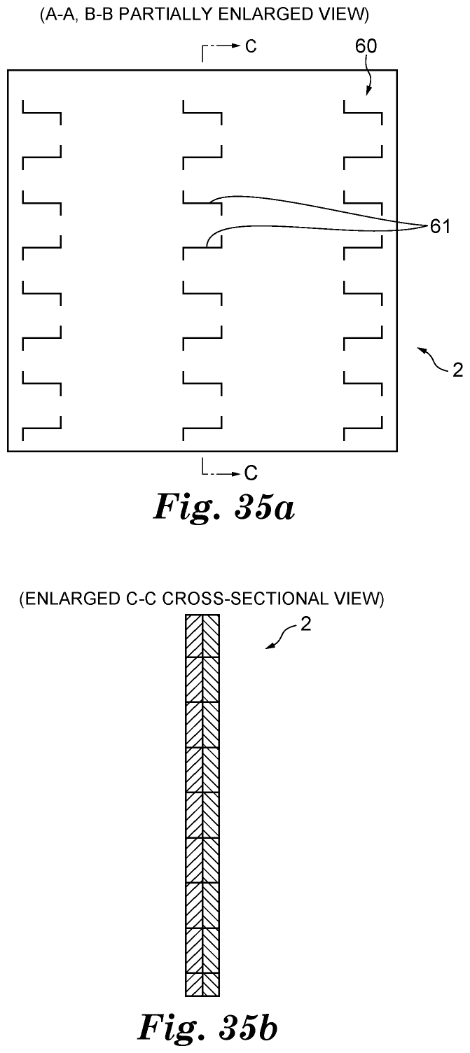

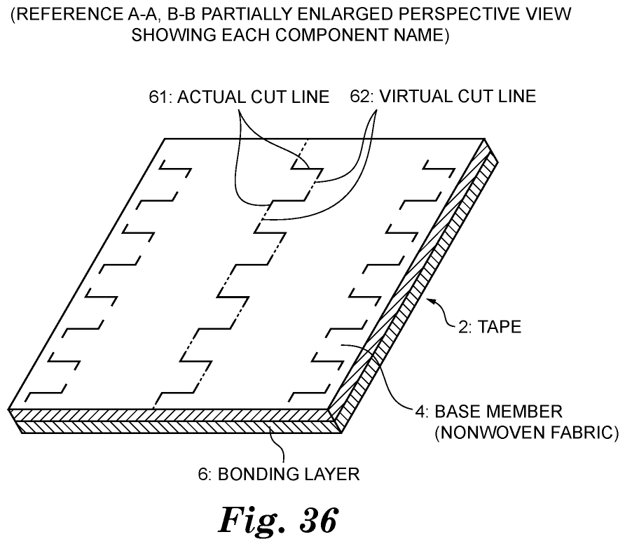

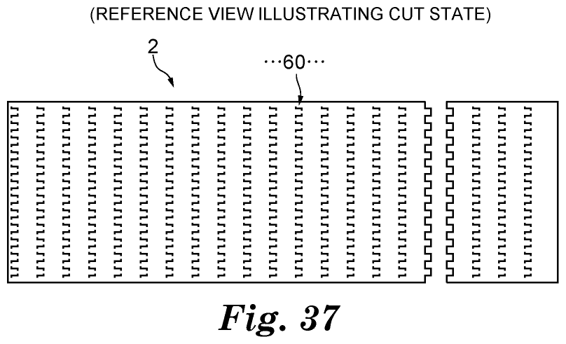

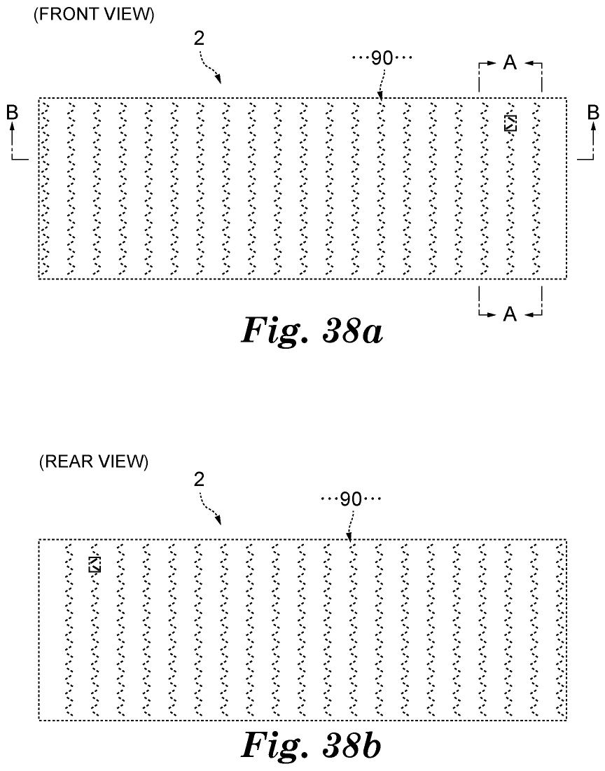

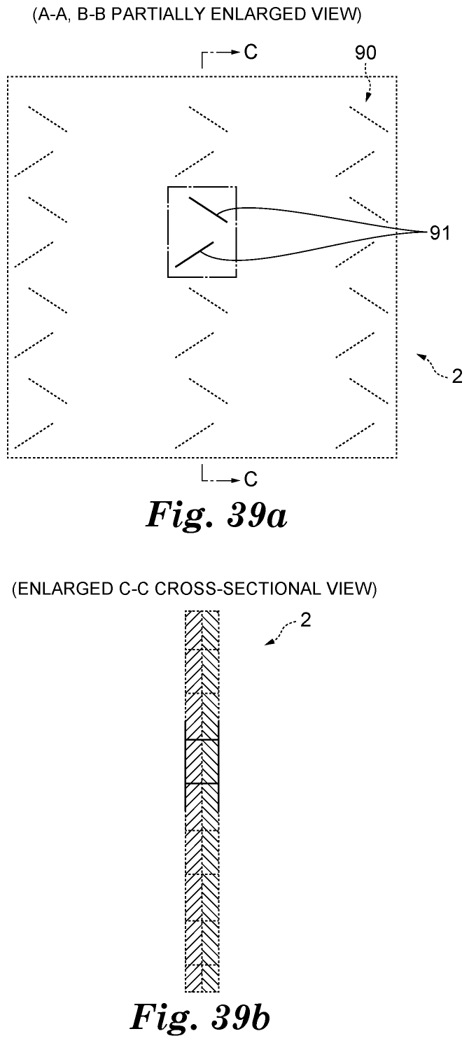

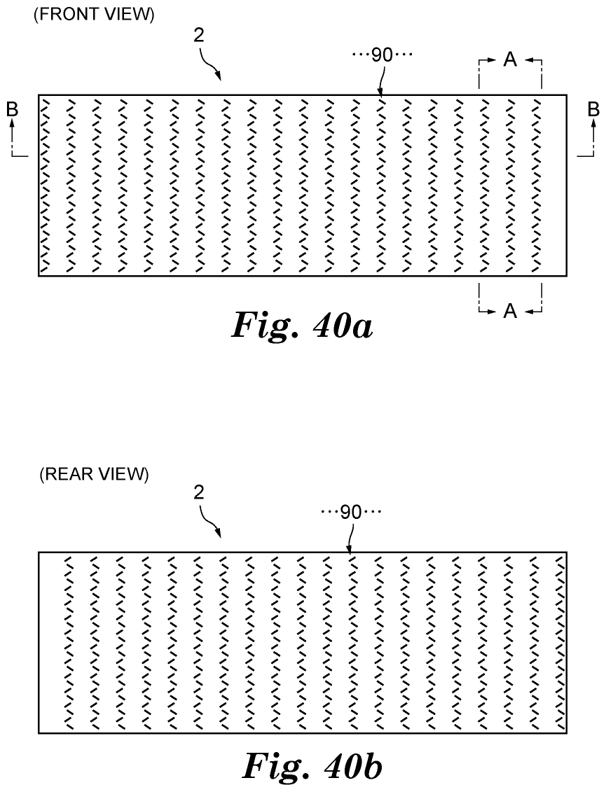

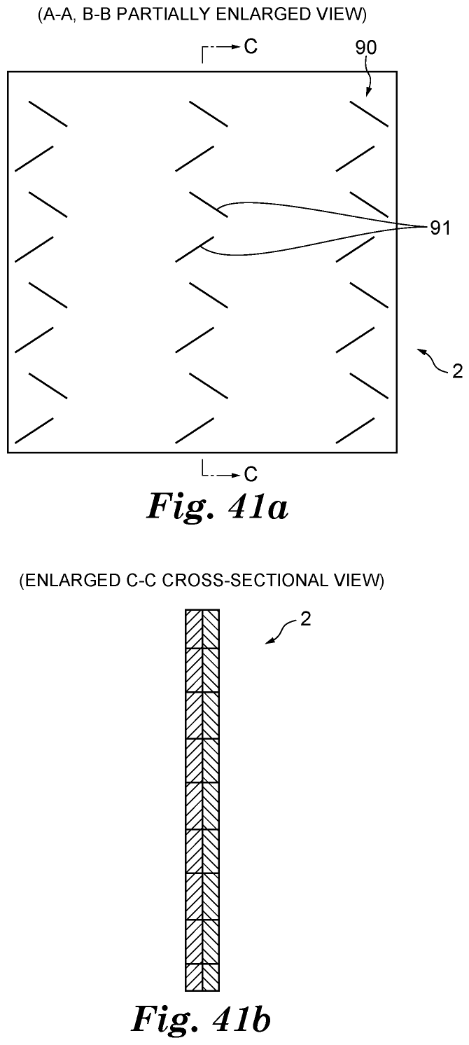

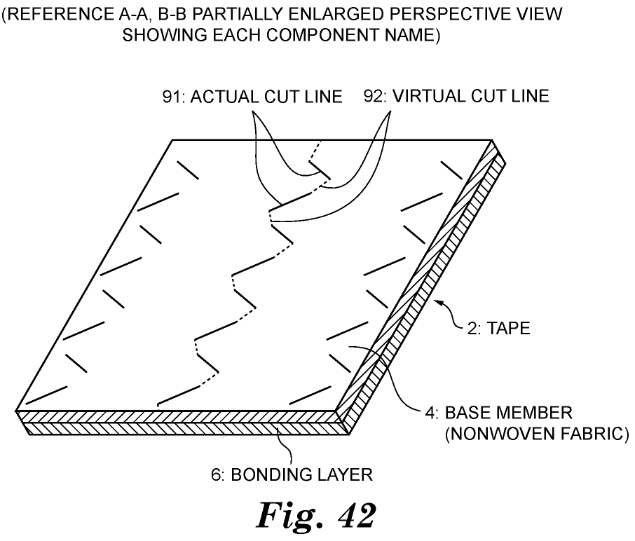

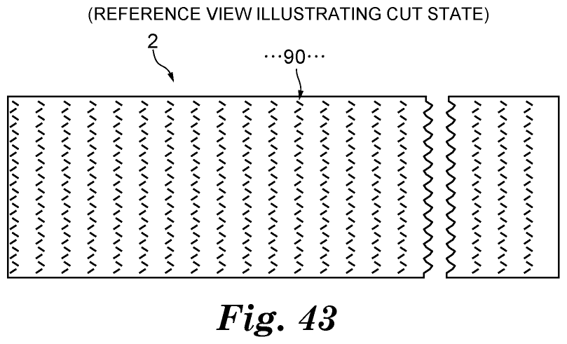

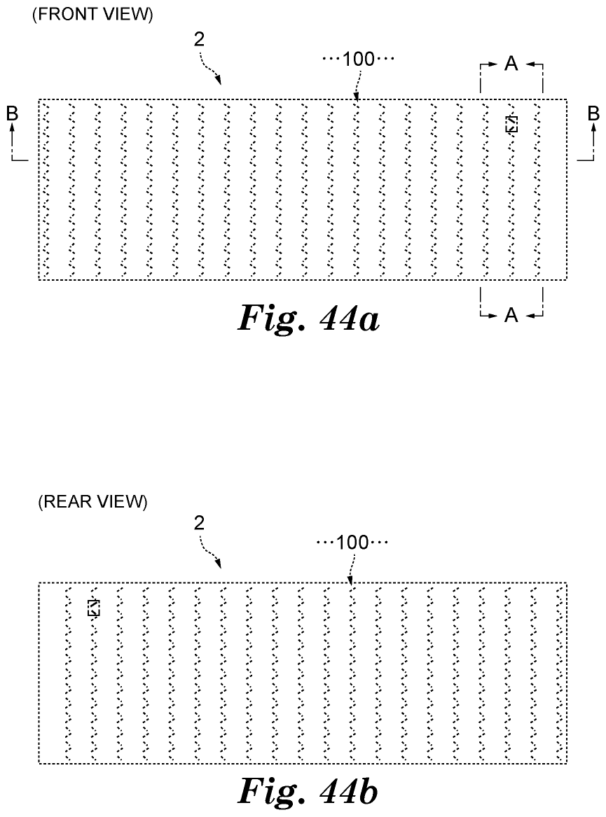

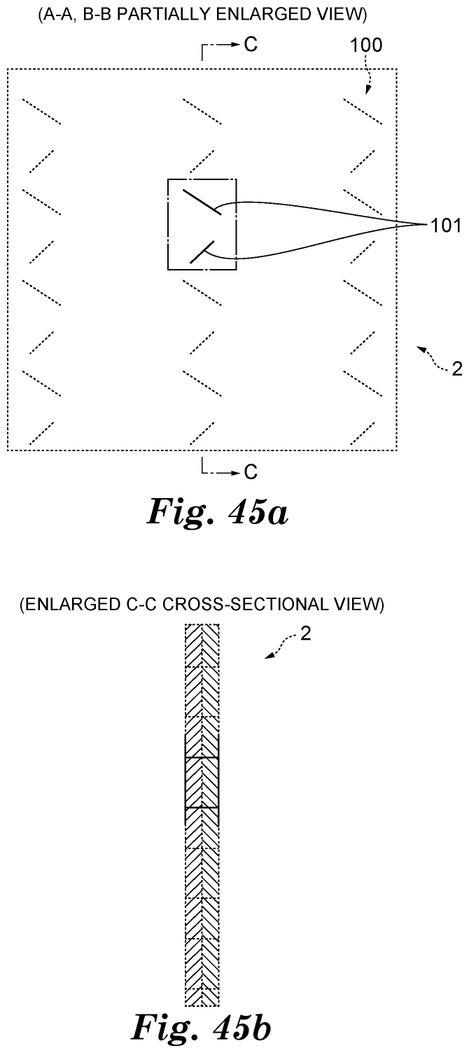

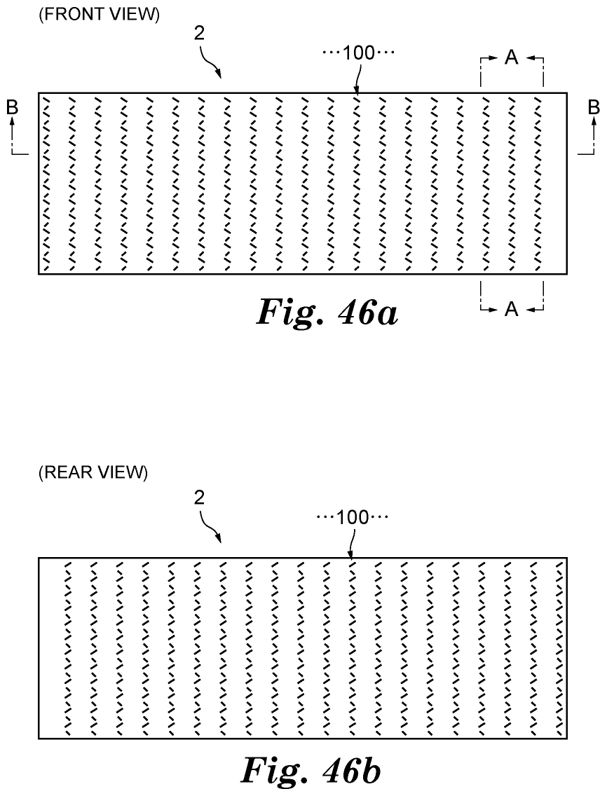

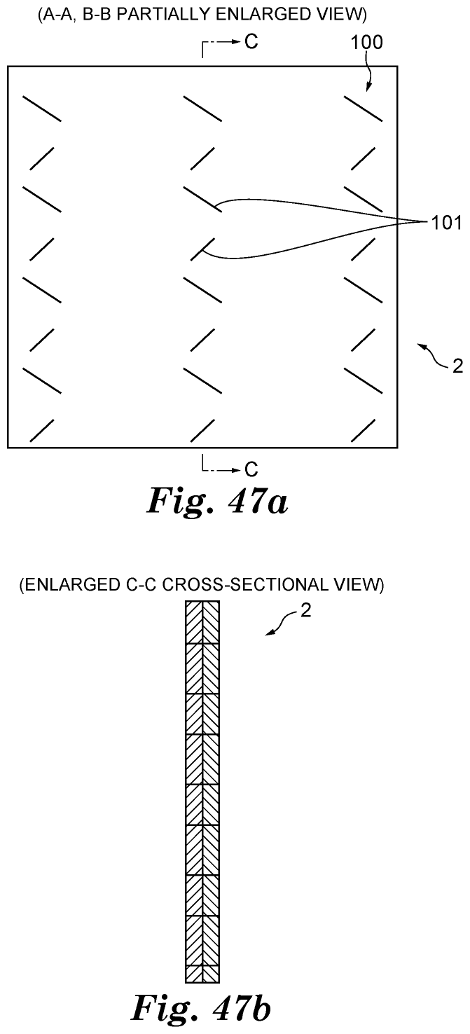

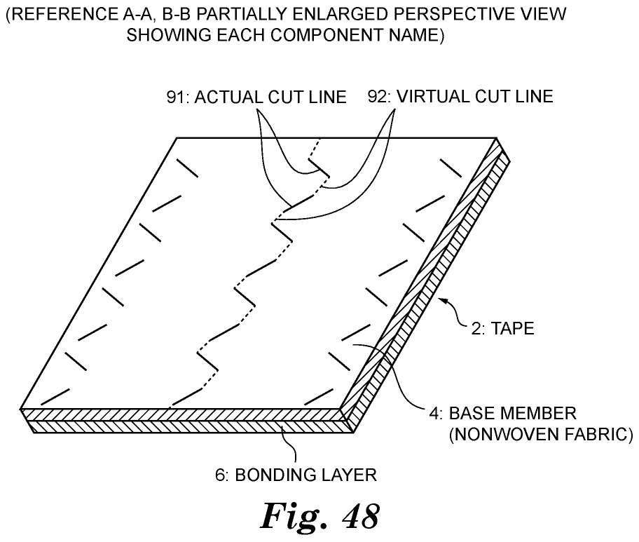

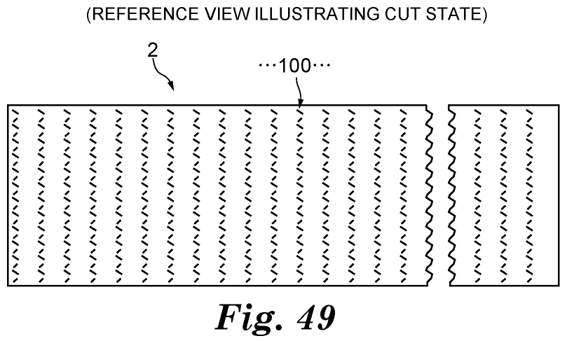

FIGS. 14A to 18 illustrate various views of the tape 2 including the cutting induction portion 10 illustrated in FIGS. 4A and 4B. FIGS. 19A to 25 illustrate various views of the tape 2 including the cutting induction portion 80 illustrated in FIG. 8. FIGS. 26A to 31 illustrate various views of the tape 2 including the cutting induction portion 70 illustrated in FIG. 7B. FIGS. 32A to 37 illustrate various views of the tape 2 including the cutting induction portion 60 illustrated in FIG. 7A. FIGS. 38A to 43 illustrate various views of the tape 2 including the cutting induction portion 90 illustrated in FIG. 10A. FIGS. 44A to 49 illustrate various views of the tape 2 including the cutting induction portion 100 illustrated in FIG. 10B.

An article illustrated in each of FIGS. 14A to 49 is used as an adhesive tape for medical use or athletic use, for example. The article is formed by applying a bonding layer to one surface of a base member including nonwoven fabric. As illustrated in the “reference A-A, B-B enlarged perspective view showing each component name,” actual cut lines penetrating the base material and the bonding layer are formed, and the actual cut lines in a combination of an inverted V-like shape are arranged as a unit at a predetermined interval in the short direction of the tape. The shortest distance between ends of actual cut lines adjacent to each other in an inverted V-like shape is a virtual cut line, and when the article is cut, the article is cut along the actual cut line and the virtual cut line as illustrated in the “reference view illustrating a cut state.” In the article, the virtual cut lines are arranged at an interval in the longitudinal direction of the tape, and thus ease of tearing is achieved while tensile strength of the tape is maintained. For example, the article is wound in the form of a roll or stacked in the form of a sheet for storage.

### REFERENCE NUMERALS

1 Roll product
2 Tape (Band-shaped member)
4 Base member
6 Bonding layer
10, 60, 70, 80, 90, 100, 110, 120 Cutting induction portion
11, 61, 71, 81, 91, 101, 111, 121 Actual cut line
12, 62, 72, 82, 92, 102, 112, 122 Virtual cut line
14, 94, 104, 114, 124 Row
16, 96, 106 Projected portion (First projected portion)
17 Projected portion (Second projected portion)

1-6. (canceled)

7. A roll product comprising a band-shaped member having longitudinal direction and width direction, wherein the band-shaped member is wound in a roll shape along the longitudinal direction, the roll product comprises:
a plurality of cutting induction portions extending in a width direction of the band-shaped member are formed in the band-shaped member in a longitudinal direction of the band-shaped member;
each of the plurality of cutting induction portions comprises:
a plurality of actual cut lines arranged to be spaced apart from each other in the width direction, and formed by partially cutting the base member, and
a plurality of virtual cut lines set by connecting ends of the plurality of actual cut lines;
the plurality of virtual cut lines include a virtual cut line extending from a first end of a first actual cut line of the plurality of actual cut lines and connected to one of ends of a second actual cut line different from the first actual cut line, and the one of the ends of the second actual cut line is closest to the first end of the ends of the second actual cut line;
the plurality of virtual cut lines include a virtual cut line extending from a second end of the first actual cut line and connected to one of ends of a third actual cut line different from the first actual cut line and the second actual cut line, and the one of the ends of the third actual cut line is closest to the second end of the ends of the third actual cut line;
a plurality of rows of the virtual cut lines arranged in the width direction are formed in the longitudinal direction in each of the cutting induction portions;
a first projection reference line extending in the longitudinal direction is set, and a plurality of first projected portions are set by projecting the plurality of virtual cut lines in each of the cutting induction portions onto the first projection reference line, the first projected portions of the plurality of virtual cut lines belonging to the same row overlap each other, and the first projected portions of the plurality of the virtual cut lines belonging to one row and the first projected portions of the plurality of the virtual cut lines belonging to another row are spaced apart in the longitudinal direction without overlapping each other; and
a second projection reference line extending in the width direction is set, and a plurality of second projected portions are set by projecting the plurality of virtual cut lines in each of the cutting induction portions onto the second projection reference line, the plurality of second projected portions are spaced apart in the width direction without overlapping each other.

8. The roll product according to claim 7, wherein two rows of the virtual cut lines are formed in each of the cutting induction portions.

9. The roll product according to claim 7, wherein the actual cut line is inclined from one side to another side in the width direction toward one side in the longitudinal direction and wherein an adjacent actual cut line is inclined from the other side to the one side in the width direction toward the one side in the longitudinal direction are alternately arranged in the width direction.

10. The roll product according to claim 7, wherein the virtual cut line extends in parallel with the width direction.

11. The roll product according to claim 7, wherein the actual cut line is a straight or curved line.

12. The roll product according to claim 7, wherein the virtual cut line is nonparallel with the width direction.

13. The roll product according to claim 7, wherein the band-shaped member comprises an adhesive coated surface.
